SEQUENCE LISTING (25 sequences)

SEQ ID NO: 1 - DNA, 41 bp, Homo sapiens
  source 1..41: mol_type = genomic DNA
ttttgtatttttagtagagayggggtttcacagtgttggtt

SEQ ID NO: 2 - DNA, 41 bp, Homo sapiens
  source 1..41: mol_type = genomic DNA
ggactccctctcaaaaaatanatacatacatacatacatac

SEQ ID NO: 3 - DNA, 41 bp, Homo sapiens
  source 1..41: mol_type = genomic DNA
  variation 21: note = n is g or del
ctgatctatttctgttttatngaactaaagcttgatttagt

SEQ ID NO: 4 - DNA, 41 bp, Homo sapiens
  source 1..41: mol_type = genomic DNA
tacatcaaaataagtcaccahgtgggcatcatgaaaccatg

SEQ ID NO: 5 - DNA, 41 bp, Homo sapiens
  source 1..41: mol_type = genomic DNA
gtcattagattatactctccdgaaagctgatttgattgatt

SEQ ID NO: 6 - DNA, 41 bp, Homo sapiens
  source 1..41: mol_type = genomic DNA
  variation 21: note = n is del or t
atttgggttttatttttaaangctgttggcatccgtgtaga

SEQ ID NO: 7 - DNA, 41 bp, Homo sapiens
  source 1..41: mol_type = genomic DNA
tttgtacacacatgttcataragcattattcacaatagcca

SEQ ID NO: 8 - DNA, 41 bp, Homo sapiens
  source 1..41: mol_type = genomic DNA
cagatagtaaattgcatgtgyattctaccacaatttaaaaa

SEQ ID NO: 9 - DNA, 41 bp, Homo sapiens
  source 1..41: mol_type = genomic DNA
  variation 21: note = n is del or tag
gaactgagcatggcagatcanattaataatgctagctgctg

SEQ ID NO: 10 - DNA, 41 bp, Homo sapiens
  source 1..41: mol_type = genomic DNA
ttaatctggaacatgtgccakcctttctttaatgaattgtg

SEQ ID NO: 11 - DNA, 41 bp, Homo sapiens
  source 1..41: mol_type = genomic DNA
gtagtagaatgaagtagaggrgacggtcacacaaacccaga

SEQ ID NO: 12 - DNA, 41 bp, Homo sapiens
  source 1..41: mol_type = genomic DNA
tagaatgaagtagaggggacrgtcacacaaacccagagggc

SEQ ID NO: 13 - DNA, 41 bp, Homo sapiens
  source 1..41: mol_type = genomic DNA
ctgaggtcaggagttcgagamcagcctggccaacatggcaa

SEQ ID NO: 14 - DNA, 41 bp, Homo sapiens
  source 1..41: mol_type = genomic DNA
acctggtggaggggagaggcdggagttgcagtgagccaaga

SEQ ID NO: 15 - DNA, 41 bp, Homo sapiens
  source 1..41: mol_type = genomic DNA
ggcaggagttgcagtgagccdagatgatgccacagcactcc

SEQ ID NO: 16 - DNA, 41 bp, Homo sapiens
  source 1..41: mol_type = genomic DNA
ccaaagacatattagagataygggaattatctacatagagg

SEQ ID NO: 17 - DNA, 41 bp, Homo sapiens
  source 1..41: mol_type = genomic DNA
aaagacatattagagatacgvgaattatctacatagagggt

SEQ ID NO: 18 - DNA, 41 bp, Homo sapiens
  source 1..41: mol_type = genomic DNA
ggtggaggactaataagaacmcattgtttcagaggccaagg

SEQ ID NO: 19 - DNA, 41 bp, Homo sapiens
  source 1..41: mol_type = genomic DNA
  variation 21: note = n is del or gag
gcacttttccctgggtcagcnaacagcaccttatcagcacc

SEQ ID NO: 20 - DNA, 41 bp, Homo sapiens
  source 1..41: mol_type = genomic DNA
  variation 21: note = n is a, del, aa, aaa, or aaaa
gccttttattgccaagtattnaaaaaaaaaacaaaaaactt

SEQ ID NO: 21 - DNA, 41 bp, Homo sapiens
  source 1..41: mol_type = genomic DNA
tttgggtgaagcttgattttmtcaccatcattctttctttt

SEQ ID NO: 22 - DNA, 41 bp, Homo sapiens
  source 1..41: mol_type = genomic DNA
aatgagagatctgggcggggraaggggagaaagatattcct

SEQ ID NO: 23 - DNA, 41 bp, Homo sapiens
  source 1..41: mol_type = genomic DNA
tgaaacttggtctcttggtchaaaagcttcctttctttata

SEQ ID NO: 24 - DNA, 41 bp, Homo sapiens
  source 1..41: mol_type = genomic DNA
tgacttgcccaaaattacacdaaatggttacagcgggacca

SEQ ID NO: 25 - DNA, 41 bp, Homo sapiens
  source 1..41: mol_type = genomic DNA
ctcaggttcctgcagctacavgtgaagtcctgaggtcccct

(19) United States
(12) Patent Application Publication
(10) Pub. No.: US 2024/0417801 A1
(43) Pub. Date: Dec. 19, 2024

(54) COMPOSITIONS AND METHODS FOR ASSESSING THE EFFICACY OF POLYNUCLEOTIDE DELIVERY AND CANCER THERAPY

(71) Applicant: Denovo Biopharma, LLC, San Diego, CA (US)

(72) Inventors: Darin Taverna, San Diego, CA (US); Hong Sun, San Diego, CA (US); Haiping Lu, San Diego, CA (US); Wen Luo, San Diego, CA (US)

(21) Appl. No.: 18/670,956

(22) Filed: May 22, 2024

Related U.S. Application Data
(63) Continuation of application No. PCT/US22/80283, filed on Nov. 22, 2022.
(60) Provisional application No. 63/282,649, filed on Nov. 23, 2021.

(51) Int. Cl. C12Q 1/6886; C12Q 1/6874

(52) U.S. Cl. CPC C12Q 1/6886; C12Q 1/6874; C12Q 2600/106; C12Q 2600/156

(57) ABSTRACT

The present invention relates to the field of pharmacogenomics, which applies one or more genomic biomarkers and the related diagnostic methods, devices, reagents, systems, and kits, for predicting varied individual responses such as, for example, efficacy or adverse effect, to gene delivery or therapy treatment using a viral vector and/or therapeutic agents, e.g., anti-neoplasm, anti-cancer, or anti-tumor drugs or prodrugs such as 5-fluorocytosine (5-FC).

### CROSS REFERENCE TO RELATED APPLICATIONS

This application claims priority to U.S. Provisional Application No. 63/282,649, filed 23 Nov. 2021 and PCT Application No. PCT/US22/80283, filed 22 Nov. 2022, the contents of which applications are incorporated herein by reference in their entireties for all purposes.

### REFERENCE TO AN ELECTRONIC SEQUENCE LISTING

The contents of the electronic sequence listing (DB-107-PCT 14Oct22.xml; Size: 25160 B; and Date of Creation: 14 Nov. 2022) is herein incorporated by reference in its entirety.

### TECHNICAL FIELD

The present invention relates to the field of pharmacogenomics, which applies one or more genomic biomarkers and the related diagnostic methods, devices, reagents, systems, and kits, for predicting varied individual responses such as, for example, efficacy or adverse effect, to gene delivery or therapy treatment using a viral vector and/or therapeutic agents, e.g., anti-neoplasm, anti-cancer, or anti-tumor drugs or prodrugs such as 5-fluorocytosine (5-FC).

### BACKGROUND

Pharmacogenomics is the study of inheritable traits affecting subject response to drug treatment. Differential responses to drug treatment may be due to underlying genetic polymorphisms (genetic variations sometimes called mutations) that affect drug metabolism. Testing subjects for these genetic polymorphisms may help to prevent adverse drug reactions and facilitate appropriate drug dosing regimens.

In the clinical setting, pharmacogenomics may enable physicians to select the appropriate pharmaceutical agents, and the appropriate dosage of these agents, for each individual subject. That is, pharmacogenomics can identify those subjects with the right genetic makeup to respond to a given therapy. In addition, pharmacogenomics can identify those subjects with genetic variations in the genes that control the metabolism of pharmaceutical compounds, so that the proper treatment (or no treatment) decision can be made, and the proper dosage can be administered.

Cancer is a disease with extensive heterogeneity. Although conventional histological and clinical features may correlate to cancer prognosis, the same apparent prognostic type of tumors varies widely in its responsiveness to therapy and consequent survival of the patient. New prognostic and predictive markers, which would facilitate an individualization of therapy for each patient, are needed to accurately predict patient response to treatments, such as small molecule or biological molecule drugs, in the clinic. The problem may be solved by the identification of new parameters that could better predict the patient's sensitivity to treatment. The classification of patient samples is a crucial aspect of cancer diagnosis and treatment. The association of a patient's response to a treatment with molecular and genetic markers can open up new opportunities for treatment development in non-responding patients, or distinguish a treatment's indication among other treatment choices because of higher confidence in the efficacy. Further, the pre-selection of patients who are likely to respond well to a therapy, treatment, medicine, drug, prodrug, or combination therapy may reduce the number of patients needed in a clinical study or accelerate the time needed to complete a clinical development program. The ability to determine which patients are responding to therapies or predict drug sensitivity in patients is particularly challenging because drug responses reflect not only properties intrinsic to the target cells, but also a host's metabolic properties. Efforts to use genetic information to predict or monitor therapy, treatment, or drug response have primarily focused on individual genes that have broad effects, such as the multidrug resistance genes mdrl and mrpl.

There is a need for new and alternative compositions and methods to determine therapy, treatment, drug sensitivity or monitor response in patients to allow the development of individualized treatment for diseases and disorders based on patient response at a molecular level. Pharmacogenomics may be used to discover and/or develop new and improved compositions and methods for gene delivery or therapy treatment using a viral vector and/or therapeutic agents, e.g., anti-neoplasm, anti-cancer, or anti-tumor drugs or prodrugs such as 5-fluorocytosine (5-FC). The present disclosure meets this and the related needs.

### SUMMARY

The summary is not intended to be used to limit the scope of the claimed subject matter. Other features, details, utilities, and advantages of the claimed subject matter will be apparent from the detailed description including those aspects disclosed in the accompanying drawings and in the appended claims.

In an aspect, the invention involves one or more genomic biomarkers that correlate with different responses (e.g., efficacy, adverse effect, and other end points) among patients receiving a treatment regime using polynucleotide or gene delivery or therapy treatment using a viral vector and/or therapeutic agents. The biomarker or biomarkers can be used in companion diagnostic tests that can help to predict polynucleotide or gene delivery or therapy, drug, or prodrug responses, and apply the polynucleotide or gene delivery or therapy treatment, drug, or prodrug only to those who will be benefited, and/or exclude those who might have negative outcome and/or adverse effects due to the treatment.

Another aspect of the invention involves an isolated polynucleotide comprising, consisting of, or consisting essentially of SNP selected from the group consisting of rs61530665, a SNP (or SNPs) in linkage disequilibrium with rs61530665, a SNP (or SNPs) in linkage disequilibrium with rs61530665, and a complementary SNP (or SNPs) thereof (e.g., a complementary SNP of rs61530665, a complementary SNP of a SNP (or SNPs) in linkage disequilibrium with rs61530665, or a combination thereof).

Another aspect of the invention involves a panel of isolated polynucleotides comprising, consisting of, or consisting essentially of two or more, three or more, four or more, or five or more of the above-described isolated polynucleotides.

Another aspect of the invention involves a kit comprising the above-described isolated polynucleotide or panel, which kit optionally comprises an instruction for use.

Another aspect of the invention involves a microarray comprising a substrate and the above-described isolated polynucleotide or panel directly or indirectly immobilized on the substrate.

Another aspect of the invention involves a kit comprising the above-described microarray, which kit optionally comprises an instruction for use.

Another aspect of the invention involves a reagent for detecting one or more SNPs selected from the group consisting of rs61530665, a SNP (or SNPs) in linkage disequilibrium with rs61530665, and a complementary SNP (or SNPs) thereof.

Another aspect of the invention involves a kit comprising the above-described reagent, which kit optionally comprises an instruction for use.

Another aspect of the invention involves a kit comprising the above-described isolated polynucleotide or panel thereof and the above-described reagent, which kit optionally comprises an instruction for use.

Another aspect of the invention involves a microarray comprising a substrate and the above-described reagent directly or indirectly immobilized on the substrate.

Another aspect of the invention involves a microarray comprising a substrate and the above-described isolated polynucleotide or panel thereof and the above-described reagent directly or indirectly immobilized on the substrate.

Another aspect of the invention involves a method, comprising:
a) assaying a biological sample from a subject that is undergoing a treatment or is considered for a treatment for one or more SNPs selected from the group consisting of rs61530665, a SNP (or SNPs) in linkage disequilibrium with rs61530665, and a complementary SNP (or SNPs) thereof; and
b) generating an output based on the assay results of the SNP or SNPs.

Another aspect of the invention involves using the output of above method to:
a) determine the likely responsiveness of the subject to the treatment; and/or
b) classify (and/or select) the subject as eligible or ineligible for the treatment or continued treatment; and/or
c) determine whether the subject or the population (when samples from a population of subjects are assayed) is likely to benefit from the treatment or continued treatment, and/or whether the subject or the population is likely to experience an adverse effect from the treatment or continued treatment; and/or
d) determine whether the subject should continue to receive the treatment.

Another aspect of the invention involves a method of treating comprising administering an effective amount of an anti-neoplasm, anti-cancer, or anti-tumor prodrug and delivering an effective amount of an agent that activates the anti-neoplasm, anti-cancer, or anti-tumor prodrug using a viral vector to a subject in need thereof and has at least one minor allele (or possesses genotypes with at least one minor allele) for one or more SNPs selected from the group consisting of rs61530665, a SNP (or SNPs) in linkage disequilibrium with rs61530665, and a complementary SNP (or SNPs) thereof.

Another aspect of the invention involves the use of an effective amount of:
a) an anti-neoplasm, anti-cancer, or anti-tumor prodrug, e.g., a pyrimidine analog, 5-fluorocytosine (5-FC), or a 5-FC analogue or derivative; and
b) a polypeptide having cytosine deaminase activity, or a viral vector configured for delivering a polynucleotide encoding a polypeptide having cytosine deaminase activity or a cytosine deaminase,
   for the manufacture of a medicament for treating a neoplasm, cancer, or a tumor in a subject in need thereof and has at least one minor allele (or possesses genotypes with at least one minor allele) for one or more SNPs selected from the group consisting of rs61530665, a SNP (or SNPs) in linkage disequilibrium with rs61530665, and a complementary SNP (or SNPs) thereof.

Another aspect of the invention involves a method for delivering a payload to a subject, which method comprises delivering an effective amount of a payload, using a viral vector that comprises a polynucleotide encoding the payload, to a subject in need and has at least one minor allele (or possesses genotypes with at least one minor allele) for one or more SNPs selected from the group consisting of rs61530665, a SNP (or SNPs) in linkage disequilibrium with rs61530665, and a complementary SNP (or SNPs) thereof.

Another aspect of the invention involves the use of an effective amount of a viral vector that comprises a polynucleotide encoding a payload for the manufacture of a medicament for treating or preventing a disease or a disorder in a subject in need thereof or prevention and has at least one minor allele (or possesses genotypes with at least one minor allele) for one or more SNPs selected from the group consisting of rs61530665, a SNP (or SNPs) in linkage disequilibrium with rs61530665, and a complementary SNP (or SNPs) thereof.

### BRIEF DESCRIPTION OF THE DRAWINGS

FIG. 1 depicts an exemplary Toca 5 phase 3 trial overall survival result. Toca's anti-tumor activity in GBM was examined in a phase 3 trial where 403 patients were randomized in 1:1 ratio in Toca treatment arm vs SOC control arm. The Kaplan-Meier estimate, also known as the product limit estimate, is a non-parametric statistic used to estimate the survival function from lifetime data. Plot based on such estimate method is called Kaplan-Meier plot. Kaplan-Meier technique is widely used in medical and clinical research to analyze time to event variables, such overall survival and disease-free survival. These time to event variables often do not satisfy an underline specific survival function, such as exponential or Weibull distribution. Therefor Kaplan-Meier technique provides a practical and least-bias estimate of survival function for common time to event variables.

FIG. 2 depicts an exemplary DB107 biomarker study design using samples of the subjects from the DB107 arm.

FIG. 3 depicts an exemplary overall survival analysis of the DB107 arm from the discovery set, comparing DGM7+ group vs. DGM7− group (DGM7+ patients possessing CT or TT genotypes for SNP rs61530665).

FIG. 4 depicts an exemplary overall survival analysis of DB107 arm from the replication set, comparing DGM7+ group vs. DGM7− group.

FIG. 5 depicts an exemplary overall survival analysis of (standard of care) SOC arm, comparing DGM7+ group vs. DGM7− group.

FIG. 6A depicts an exemplary overall survival analysis of DGM7+ objects, comparing DB107 arm vs. SOC arm. FIG. 6B depicts an exemplary overall survival analysis of the following four groups: DGM7+ subjects in DB107 arm, DGM7− subjects in DB107 arm, DGM7+ subjects in SOC arm, and DGM7− subjects in SOC arm.

FIG. 7 depicts an exemplary physical location of rs61530665 on Chromosome 4 and its overlapping gene SHROOM3.

### DETAILED DESCRIPTION OF THE INVENTION

A detailed description of one or more aspects of the claimed subject matter is provided below along with accompanying figures that illustrate the principles of the claimed subject matter. The claimed subject matter is described in connection with such aspects but is not limited to any aspect. It is to be understood that the claimed subject matter may be embodied in various forms and encompasses numerous alternatives, modifications, and equivalents. Therefore, specific details described herein are not to be interpreted as limiting, but rather as a basis for the claims and as a representative basis for teaching one skilled in the art to employ the claimed subject matter in virtually any appropriately detailed system, structure, or manner. Numerous specific details are set forth in the following description to provide a thorough understanding of the present disclosure. These details are provided for the purpose of example and the claimed subject matter may be practiced according to the claims without some or all these specific details. It is to be understood that other aspects can be used, and structural changes can be made, without departing from the scope of the claimed subject matter. The various features and functionality described in one or more of the individual aspects are not limited in their applicability to the aspect with which they are described. They instead can, be applied, alone or in some combination, to one or more of the other aspects of the disclosure, whether such aspects are described, and whether such features are presented as being a part of a described aspect. To clarify, technical material that is known in the technical fields related to the claimed subject matter has not been described in detail so that the claimed subject matter is not unnecessarily obscured.

All publications referred to in this application are incorporated by reference in their entireties for all purposes to the same extent as if each individual publication were individually incorporated by reference.

All headings are for the convenience of the reader and should not be used to limit the meaning of the text that follows the heading, unless so specified.

Throughout this disclosure, various aspects of the claimed subject matter are presented in a range format. The description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6.

### A. General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994).

### B. Definitions

Unless defined otherwise, all technical and scientific terms used herein have the same meaning as is commonly understood by one of ordinary skill in the art to which this invention belongs. All patents, applications, published applications, and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

The singular forms “a”, “an”, and “the” include plural references unless indicated otherwise. For example, “a” dimer includes one or more dimers.

SNP is single nucleotide polymorphism.

SNPs is single nucleotide polymorphisms.

“Biomarker” or “marker” as used herein refers generally to a molecule, including a gene, protein, carbohydrate structure, or glycolipid, the expression of which in or on a mammalian tissue or cell or secreted can be detected by known methods (or methods described herein) and is predictive or can be used to predict (or aid prediction) for a mammalian cell's or tissue's sensitivity to, and in some aspects, to predict (or aid prediction) an individual's responsiveness to treatment regimens.

A “pharmacogenomic biomarker” is an objective biomarker which correlates with a therapy, a treatment, or a specific clinical drug response or susceptibility in a subject (see, e.g., McLeod et al., Eur. J. Cancer (1999) 35:1650-1652). It may be a biochemical biomarker or a clinical sign or symptom. The presence or quantity of the pharmacogenomic marker is related to the predicted response of the subject to a therapy, a treatment, or a specific drug or class of drugs prior to administration of the drug. By assessing the presence or quantity of one or more pharmacogenomic markers in a subject, a therapy, a treatment, or a drug (prodrug) therapy which is most appropriate for the subject, or which is predicted to have a greater degree of success, may be selected. For example, based on the presence or quantity of DNA, RNA, or protein for specific tumor markers in a subject, a therapy, a treatment, a drug, or course of treatment may be selected that is optimized for the treatment of a disease or a disorder, e.g., a neoplasm, cancer, or tumor, likely to be present in the subject. Similarly, the presence or absence of a specific sequence mutation or polymorphism may correlate with therapy, treatment or drug response. The use of pharmacogenomic biomarkers therefore permits the application of the most appropriate treatment for each subject without having to administer the therapy. Methods for discovering pharmacogenomic biomarkers are known, for example, as disclosed in US 2014/0031242 A1 and US 2015/0368720 A1, which are incorporated herein by reference.

The term “polymorphic locus” refers to a region in a nucleic acid at which two or more alternative nucleotide sequences are observed in a significant number of nucleic acid samples from a population of individuals. A polymorphic locus may be a nucleotide sequence of two or more nucleotides, an inserted nucleotide or nucleotide sequence, a deleted nucleotide or nucleotide sequence, or a microsatellite, for example. A polymorphic locus that is two or more nucleotides in length includes sequences of 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic locus is often one nucleotide in length, which is referred to herein as a “single nucleotide polymorphism” or a “SNP.” In some aspects, high-density genotyping may be conducted by using SNPs. In some aspects, about 1,000-5,000,000 or more SNPs, may be used. In some aspects, high-density genotyping may be array-based. In some aspects, high-density genotyping may be conducted by using sequencing, such as high-throughput sequencing.

Where there are two, three, or four alternative nucleotide sequences at a polymorphic locus, each nucleotide sequence is referred to as a “polymorphic variant” or “nucleic acid variant.” Where two polymorphic variants exist, for example, the polymorphic variant represented in a minority of samples from a population is referred to as a “minor allele” and the polymorphic variant that is more prevalently represented is referred to as a “major allele.” Many organisms possess a copy of each chromosome (e.g., humans), and those individuals who possess two major alleles or two minor alleles are to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele.

Single-nucleotide polymorphisms (SNPs) may fall within coding sequences of genes, non-coding regions of genes, or in the intergenic regions (regions between genes). SNPs within a coding sequence do not necessarily change the amino acid sequence of the protein that is produced, due to degeneracy of the genetic code.

SNPs in the coding region are of two types, synonymous and nonsynonymous SNPs. Synonymous SNPs do not affect the protein sequence while nonsynonymous SNPs change the amino acid sequence of protein. The nonsynonymous SNPs are of two types: missense and nonsense. SNPs that are not in protein-coding regions may still affect gene splicing, transcription factor binding, messenger RNA degradation, or the sequence of non-coding RNA. Gene expression affected by this type of SNP is referred to as an eSNP (expression SNP) and may be upstream or downstream from the gene.

In genetic analysis that identifies one or more pharmacogenomic biomarkers, samples from individuals having different values in a relevant phenotype often are allelotyped and/or genotyped. The term “allelotype” refers to a process for determining the allele frequency for a polymorphic variant in pooled DNA samples from cases and controls, and/or in separate DNA samples from each individual subject. By genotyping DNA from each group, an allele frequency for each locus in each group is calculated. These allele frequencies are then compared to one another. In some aspects, DNA samples are genotyped using whole genome SNP arrays, such as those manufactured by Affymetrix (Santa Clara, Calif.) and/or Illumina (San Diego, Calif.), such as the Affymetrix 500K array. In addition to Affymetrix arrays, Illumina chips, and Sequenom MassArray can also be used. Any suitable genotype calling algorithm(s) may be used. In some aspects, the genotype calls are generated using the Robust Linear Model with the Mahalanobis Distance Classifier (RLMM) algorithm, the RLMM with a Bayesian step (BRLMM) algorithm, the Axiom™ GT1 algorithm, the BRLMM using perfect-match probes (BRLMM-P) algorithm, or the Birdseed algorithm (Rabbee et al., Bioinformatics (2006) 22:7-12; Korn et al., Nat Genet (2008) 40:1253-60).

A genotype or polymorphic variant may be expressed in terms of a “haplotype,” which refers to a set of DNA variations, or polymorphisms, that tend to be inherited together. A haplotype can refer to a combination of alleles or to a set of SNPs found on the same chromosome. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

Sometimes, a polymorphic variant is reported in a database without determining whether the variant is represented in a significant fraction of a population. Because a subset of these reported polymorphic variants are not represented in a statistically significant portion of the population, some of them are sequencing errors and are not biologically relevant. Thus, it is often not known whether a reported polymorphic variant is statistically significant or biologically relevant until the presence of the variant is detected in a population of individuals and the frequency of the variant is determined. A polymorphic variant is statistically significant (and optionally often biologically relevant) if it is represented in 1% or more of a population, sometimes 5% or more, 10% or more, 15% or more, or 20% or more of a population, and often 25% or more, 30% or more, 35% or more, 40% or more, 45% or more, or 50% or more of a population. For certain genetic diseases and/or rare diseases, however, a variant may represent a very small percentage of a population and yet is still biologically relevant.

The term “sample” refers to a composition that is obtained or derived from a subject of interest that contains a cellular and/or other molecular entity that is to be characterized and/or identified, for example based on physical, biochemical, chemical, and/or physiological characteristics. For example, the phrase “clinical sample” or “disease sample” and variations thereof refer to any sample obtained from a subject of interest that would be expected or is known to contain the cellular and/or molecular entity that is to be characterized.

The term “tissue or cell sample” refers to a collection of similar cells obtained from a tissue of a subject or patient. The source of the tissue or cell sample may be solid tissue as from a fresh, frozen, and/or preserved organ or tissue sample or biopsy or aspirate; blood or any blood constituents; bodily fluids such as cerebral spinal fluid, amniotic fluid, peritoneal fluid, or interstitial fluid; and/or cells from any time in gestation or development of the subject. The tissue sample may also be primary or cultured cells or cell lines. Optionally, the tissue or cell sample is obtained from a disease tissue/organ. The tissue sample may contain compounds that are not naturally intermixed with the tissue in nature such as preservatives, anticoagulants, buffers, fixatives, nutrients, antibiotics, or the like.

The biological sample can be a plasma, serum, whole blood, or dried blood spot sample. “Plasma” or “blood plasma” refers to the intravascular fluid part of extracellular fluid (all body fluid outside of cells). It is mostly water and contains dissolved proteins, glucose, clotting factors, mineral ions, hormones, and carbon dioxide (plasma being the main medium for excretory product transportation). Blood plasma is prepared by spinning a tube of fresh blood containing an anti-coagulant in a centrifuge until the blood cells fall to the bottom of the tube. The blood plasma is then poured or drawn off. “Blood serum” is blood plasma without fibrinogen or the other clotting factors (i.e., whole blood minus both the cells and the clotting factors).

“Polynucleotide,” or “nucleic acid,” as used interchangeably herein, refer to polymers of nucleotides of any length and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, aspects wherein phosphate is replaced by P(O)S (“thioate”), P(S)S (“dithioate”), “(O)NR 2 (“amidate”), P(O)R, P(O)OR′, CO or CH 2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide” generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

“Amplification” generally refers to the process of producing multiple copies of a desired sequence. “Multiple copies” means at least 2 copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The term “array” or “microarray” refers to an ordered arrangement of hybridizable array elements, such as polynucleotide probes (e.g., oligonucleotides), beads, or binding reagents (e.g., antibodies), on a substrate. The substrate can be a solid substrate, such as a glass or silica slide, a fiber optic binder, or a semi-solid substrate, such as nitrocellulose membrane. The nucleotide sequences can be DNA, RNA, or any permutations thereof.

As used herein, the term “phenotype” refers to a trait which can be compared between individuals, such as presence or absence of a condition, a visually observable difference in appearance between individuals, metabolic variations, physiological variations, variations in the function of biological molecules, and the like. A phenotype can be qualitative or quantitative. An example of a phenotype is responsiveness to a treatment, such as a drug.

“Responsiveness” can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in lesional size; (4) inhibition (i.e., reduction, slowing down or complete stopping) of disease cell infiltration into adjacent peripheral organs and/or tissues; (5) inhibition (i.e., reduction, slowing down or complete stopping) of disease spread; (6) relief, to some extent, of one or more symptoms associated with the disorder; (7) increase in the length of disease-free presentation following treatment; (8) decreased mortality at a given point of time following treatment; and/or (9) lack of adverse effects following treatment. Responsiveness can also be assessed using any endpoint indicating side effect and/or toxicity to the patient.

“Treating” or “treatment” or “alleviation” refers to therapeutic treatment wherein the object is to slow down (lessen) if not cure the targeted pathologic condition or disorder and/or prevent recurrence of the condition. A subject is successfully “treated” if, after receiving a therapeutic amount of a therapeutic agent, the subject shows observable and/or measurable reduction in or absence of one or more signs and symptoms of the disease. For example, increase in length of remission, and/or relief to some extent, one or more of the symptoms associated with a disease or disorder, e.g., a neoplasm, cancer, or tumor; reduced morbidity and mortality, and improvement in quality of life issues. Reduction of the signs or symptoms of a disease or disorder, e.g., a neoplasm, cancer, or tumor, may also be felt by the patient. Treatment can achieve a complete response, defined as disappearance of all signs of a disease or disorder, e.g., a neoplasm, cancer, or tumor, or a partial response, preferably reduction by more than 50&, more preferably by 75% of signs of a disease or disorder. A patient is also considered treated if the patient experiences stable disease. In some aspects, treatment with a therapeutic agent is effective to result in the patients being disease-free 3 months after treatment. Additional examples include 6 months, one year, 2 or more years post treatment. These parameters for assessing successful treatment and improvement in the disease are readily measurable by routine procedures familiar to a physician of appropriate skill in the art.

The term “prediction” or “prognosis” refers to the likelihood that a patient will respond either favorably or unfavorably to a drug or set of drugs. In another aspect, the prediction relates to the extent of those responses. In another aspect, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for a patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc.

“Specifically binds” refers to the binding specificity of a specific binding pair. Recognition by an antibody of a particular target in the presence of other potential targets is one characteristic of such binding. Specific binding involves two different molecules wherein one of the molecules specifically binds with the second molecule through chemical or physical means. The two molecules are related in the sense that their binding with each other is such that they can distinguish their binding partner from other assay constituents having similar characteristics. The members of the binding component pair are referred to as ligand and receptor (anti-ligand), specific binding pair (SBP) member and SBP partner, and the like. A molecule may also be an SBP member for an aggregation of molecules; for example, an antibody raised against an immune complex of a second antibody and its corresponding antigen may be considered to be an SBP member for the immune complex.

“Homologue” is used to refer to a nucleic acid which differs from a naturally occurring nucleic acid (i.e., the “prototype” or “wild-type” nucleic acid) by minor modifications to the naturally occurring nucleic acid but which maintains the basic nucleotide structure of the naturally occurring form. Such changes include changes in one or a few nucleotides, including deletions (e.g., a truncated version of the nucleic acid) insertions and/or substitutions. A homologue can have enhanced, decreased, or substantially similar properties as compared to the naturally occurring nucleic acid. A homologue can be complementary or matched to the naturally occurring nucleic acid. Homologues can be produced using techniques known in the art to produce nucleic acids including recombinant DNA techniques, chemical synthesis, etc.

“Complementary” or “matched” means that two nucleic acid sequences have at least 50% sequence identity. Further examples include the two nucleic acid sequences having at least 60%, 70%, 80%, 90%, 95%, 96%, 97%, 98%, 99% or 100% of sequence identity. “Complementary or matched” also means that two nucleic acid sequences can hybridize under low, middle, and/or high stringency condition(s).

“Substantially complementary” or “substantially matched” means that two nucleic acid sequences have at least 90% sequence identity. Further examples include the two nucleic acid sequences having at least 95%, 96%, 97%, 98%, 99% or 100% of sequence identity. Alternatively, “substantially complementary” or “substantially matched” means that two nucleic acid sequences can hybridize under high stringency condition(s).

In general, the stability of a hybrid is a function of the ion concentration and temperature. Typically, a hybridization reaction is performed under conditions of lower stringency, followed by washes of varying, but higher, stringency. Moderately stringent hybridization refers to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions are conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C.

Low stringency hybridization refers to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA). Other suitable moderate stringency and high stringency hybridization buffers and conditions are well known to those of skill in the art.

“Output” refers to a value or score, e.g., a value or score generated from a computer algorithm. The output may be generated based on assay results using the biomarkers described herein as inputs to the computer algorithm. An “output” can be either quantitative or qualitative and can be used for determining the likely responsiveness of a subject to a treatment in a companion diagnostic test.

A companion diagnostic test or method generally provides information that is essential for the safe and effective use of a corresponding therapy, treatment, drug, prodrug, or biological product. The test helps a health care professional determine whether a particular therapy, treatment, or therapeutic product's benefits to patients will outweigh any potential serious side effects or risks. In certain aspects, a companion diagnostic test described herein can:
a) identify patients who are most likely to benefit from a particular therapy or treatment, e.g., a gene delivery or therapy treatment using a viral vector or a therapeutic agent, such as an anti-neoplasm, anti-cancer, or anti-tumor prodrug, e.g., a pyrimidine analog, 5-fluorocytosine (5-FC), or an analogue or derivative thereof;
b) identify patients likely to be at increased risk for serious side effects because of treatment with a particular therapeutic agent; and/or
c) monitor response to treatment with a particular therapeutic agent for the purpose of adjusting treatment to achieve improved safety or effectiveness.

Companion diagnostics may be co-developed with one or more drugs (or a combination therapy such as a cocktail) to aid in selecting or excluding patients (or patient groups) for/from treatment with that drug on the basis of their biological characteristics that determine responders and non-responders to the therapy. In some aspects, companion diagnostics are developed based on companion biomarker(s), biomarkers that prospectively help predict likely response and/or severe toxicity. In some aspects, there is described a companion biomarker comprising one or more SNPs described herein (for example, 1, 2, 3, 4, 5, 6, 7, 8, 9, 10, or more SNPs).

“Pharmaceutically acceptable salt” means a salt which is acceptable for administration to a patient, such as a mammal, particularly a human, i.e., salts with counterions having acceptable mammalian safety for a given dosage regime. Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

Aspects described herein include “consisting” and/or “consisting essentially of” aspects.

Other objects, advantages, and features of the present invention will become apparent from the following specification taken in conjunction with the accompanying drawings.

### C. Isolated Polynucleotides, Biomarkers, Related Compositions and Uses Thereof

In some aspects, the invention involves an isolated polynucleotide comprising, consisting of, or consisting essentially of SNP selected from the group consisting of rs61530665, a SNP (or SNPs) in linkage disequilibrium with rs61530665, and a complementary SNP (or SNPs) thereof.

In some aspects, the SNP is selected from the group consisting of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a SNP listed in any of Tables 1A-1E, 2 and 3, a complementary SNP (or SNPs) thereof, and a SNP (or SNPs) in linkage disequilibrium therewith.

In some aspects, a SNP is in linkage disequilibrium with rs61530665 when the SNP has a D′ value of linkage equilibrium equal to or greater than about 0.900 and/or a r² value between the SNP and rs61530665 being equal to or greater than about 0.800. In other aspects, a SNP is in linkage disequilibrium with rs61530665 when the D′ value of linkage equilibrium of the SNP is 1 and/or the r² value between the SNP and rs61530665 is 1, e.g., a SNP listed in Table 3, or a complementary SNP (or SNPs) thereof.

In other aspects, the SNP is rs61530665 or a complementary SNP (or SNPs) thereof.

Another aspect of the invention involves a panel of isolated polynucleotides comprising, consisting of, or consisting essentially of two or more, three or more, four or more, or five or more of the above isolated polynucleotides.

In some aspects, the panel comprises at least two of the SNPs listed in Table 3, or a complementary SNP (or SNPs) thereof, e.g., rs61530665 and at least one of the SNPs listed in Table 3, or a complementary SNP (or SNPs) thereof.

In other aspects, the panel comprises two or more, three or more, four or more, five or more, or all of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, or a complementary SNP (or SNPs) thereof.

In some aspects, the isolated polynucleotide or panel comprises isolated polynucleotide that comprises, consists of, or consists essentially of any of the sequences set forth in SEQ ID NOs: 1-25, a complementary sequence (or sequences) thereof, or a sequence (or sequences) in linkage disequilibrium therewith.

In other aspects, the isolated polynucleotide or panel comprises isolated polynucleotide that comprises, consists of, or consists essentially of any of the sequences set forth in SEQ ID NOs: 1-11, 15-21, and 25, a complementary sequence (or sequences) thereof, or a sequence (or sequences) in linkage disequilibrium therewith.

In other aspects, the isolated polynucleotide or panel comprises isolated polynucleotide that comprises, consists of, or consists essentially of any of the sequences set forth in SEQ ID NOs: 1-5, a complementary sequence (or sequences) thereof, or a sequence (or sequences) in linkage disequilibrium therewith.

In other aspects, the isolated polynucleotide or panel comprises isolated polynucleotide that comprises, consists of, or consists essentially of a sequence set forth in SEQ ID NO: 1, a complementary sequence thereof, or a sequence in linkage disequilibrium therewith.

Another aspect of the invention involves a kit comprising the above isolated polynucleotide or panel, which kit optionally comprises an instruction for use.

Another aspect of the invention involves a microarray comprising a substrate and the above isolated polynucleotide or panel directly or indirectly immobilized on the substrate.

Another aspect of the invention involves a reagent for detecting one or more SNPs selected from the group consisting of rs61530665, a SNP (or SNPs) in linkage disequilibrium with rs61530665, and a complementary SNP (or SNPs) thereof.

In some aspects, the reagent is configured for detecting rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a SNP listed in any of Tables 1A-1E, 2 and 3, a complementary SNP (or SNPs) thereof, and a SNP (or SNPs) in linkage disequilibrium therewith.

In other aspects, the reagent is configured for detecting a SNP in linkage disequilibrium with rs61530665 with the D′ value of linkage equilibrium of the SNP being 1 or with the r² value between the SNP and rs61530665 being 1, e.g., a SNP listed in Table 3, or a complementary SNP (or SNPs) thereof.

In other aspects, the present reagent is configured for detecting rs61530665, or a complementary SNP (or SNPs) thereof.

In some aspects, the SNP or SNPs in the reagent comprise any of the sequences set forth in SEQ ID NOs: 1-25, a complementary sequence (or sequences) thereof, or a sequence (or sequences) in linkage disequilibrium therewith.

In other aspects, the SNP or SNPs in the reagent comprise any of the sequences set forth in SEQ ID NOs: 1-11, 15-21, and 25, a complementary sequence (or sequences) thereof, or a sequence (or sequences) in linkage disequilibrium therewith.

In other aspects, the SNP or SNPs in the reagent comprise any of the sequences set forth in SEQ ID NOs: 1-5, a complementary sequence (or sequences) thereof, or a sequence (or sequences) in linkage disequilibrium therewith.

In aspects, the SNP or SNPs in the reagent comprise a sequence set forth in SEQ ID NO: 1, a complementary sequence thereof, or a sequence in linkage disequilibrium therewith.

In some aspects, the reagent comprises one or more molecules for assaying the SNP or SNPs. Any suitable molecule(s) can be used. For example, the one or more molecules comprise an oligonucleotide and/or a polypeptide. In some aspects, the reagent comprises oligonucleotide that comprises any of the sequences set forth in SEQ ID NOs: 1-25, or a complementary sequence (or sequences) thereof.

In other aspects, the reagent comprises oligonucleotide that comprises one or more primers for genotyping the SNP or SNPs.

Another aspect of the invention involves a kit comprising the above reagent, which kit optionally comprises an instruction for use.

Another aspect of the invention involves a kit comprising the above isolated polynucleotide or panel and the above reagent, which kit optionally comprises an instruction for use.

In some aspects, the kit comprises an isolated polynucleotide or panel that comprises a SNP selected from the group consisting of rs61530665, a SNP (or SNPs) in linkage disequilibrium with rs61530665, and a complementary SNP (or SNPs) thereof, and the reagent is capable of detecting the SNP(s).

In other aspects, the kit comprises an isolated polynucleotide or panel that comprises a SNP (or SNPs) selected from the group consisting of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a SNP listed in any of Tables 1A-1E, 2 and 3, a complementary SNP (or SNPs) thereof, and a SNP in linkage disequilibrium therewith, and the reagent is capable of detecting the SNP(s).

In other aspects, the kit comprises an isolated polynucleotide or panel that comprises a SNP that is a SNP in linkage disequilibrium with rs61530665 with the D′ value of linkage equilibrium of the SNP being 1 or with the r² value between the SNP and rs61530665 being 1, e.g., a SNP listed in Table 3, or a complementary SNP (or SNPs) thereof, and the reagent can detect the SNP(s).

In other aspects, the kit comprises isolated polynucleotide or panel that comprises a SNP that is rs61530665, or a complementary SNP (or SNPs) thereof, and the reagent can detect rs61530665.

In some aspects, in the kit, the reagent is capable of detecting the SNP(s), and the isolated polynucleotide or panel serves as a control for a detection assay.

Another aspect of the invention involves a microarray comprising a substrate and the above-described reagent directly or indirectly immobilized on the substrate.

Another aspect of the invention involves a microarray comprising a substrate and the above-described isolated polynucleotide or panel and the above-described reagent directly or indirectly immobilized on the substrate.

Another aspect of the invention involves a microarray comprising a substrate and the above-described isolated polynucleotide, panel, and/or reagent directly or indirectly immobilized on the substrate.

In some aspects, in the microarray, the reagent is capable of detecting the SNP(s) and the isolated polynucleotide or panel serves as a control for a detection assay.

In some aspects, the kit, reagent, or microarray is used for the assessment of an isolated biomarker or a panel of isolated biomarkers, wherein the biomarker or biomarkers comprise a SNP selected from the group consisting of rs61530665, a SNP (or SNPs) in linkage disequilibrium with rs61530665, and a complementary SNP (or SNPs) thereof.

In other aspects, the biomarker or biomarkers comprise a SNP (or SNPs) selected from the group consisting of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a SNP listed in any of Tables 1A-1E, 2 and 3, a complementary SNP (or SNPs) thereof, and a SNP in linkage disequilibrium therewith, and the reagent is capable of detecting the SNP(s).

In other aspects, the biomarker or biomarkers comprise a SNP that is a SNP in linkage disequilibrium with rs61530665 with the D′ value of linkage equilibrium of the SNP being 1 or with the r² value between the SNP and rs61530665 being 1, e.g., a SNP listed in Table 3, or a complementary SNP (or SNPs) thereof, and the reagent can detect the SNP(s).

In other aspects, the biomarker or biomarkers comprise a SNP that is rs61530665, or a complementary SNP (or SNPs) thereof, and the reagent can detect rs61530665.

The biomarker or a panel of the biomarkers can be assayed using any suitable assay technique or format.

Suitable assay techniques or formats include sequencing, polymerase chain reaction (PCR), capillary electrophoresis, mass spectrometry, single-strand conformation polymorphism (SSCP), electrochemical analysis, denaturing HPLC and gel electrophoresis, restriction fragment length polymorphism, hybridization analysis, single-base extension (SBE), allele specific primer extension (ASPE), restriction enzyme digestion, strand displacement amplification (SDA), transcription mediated amplification (TMA), ligase chain reaction (LCR), nucleic acid sequence based amplification (NASBA), primer extension, rolling circle amplification (RCA), self-sustained sequence replication (3SR), loop-mediated isothermal amplification (LAMP), hybridization, nucleic acid sequencing, and/or microarray. Any suitable nucleic acid sequencing technique or format can be used. For example, the nucleic acid sequencing can be selected from the group consisting of Maxam-Gilbert sequencing, a chain-termination method, shotgun sequencing, bridge PCR, single-molecule real-time sequencing, ion semiconductor (ion torrent sequencing), sequencing by synthesis, sequencing by ligation (SOLiD sequencing), chain termination (Sanger sequencing), massively parallel signature sequencing (MPSS), polony sequencing, 454 pyrosequencing, Illumina (Solexa) sequencing, DNA nanoball sequencing, heliscope single molecule sequencing, single molecule real time (SMRT) sequencing, nanopore DNA sequencing, tunnelling currents DNA sequencing, sequencing by hybridization, sequencing with mass spectrometry, microfluidic Sanger sequencing, a microscopy-based technique, RNAP sequencing, and in vitro virus high-throughput sequencing.

In some aspects, the kit, reagent, or microarray further comprises an instruction for using the isolated biomarker or panel to conduct a companion diagnostic test for a treatment, e.g., a cancer treatment.

In some aspects, the treatment can be a cancer treatment. In some aspects, the cancer is a, a brain cancer, a multiple myeloma, a pancreatic cancer, a liver cancer, a stomach cancer, a breast cancer, a kidney cancer, a lung cancer, a colorectal cancer, a colon cancer, a prostate cancer, an ovarian cancer, a cervical cancer, a skin cancer, an esophagus cancer, a head and neck cancer, a lymphoma, or a leukemia. In some aspects, the cancer is glioma/glioblastoma (GBM), anaplastic astrocytoma (AA), or other brain tumors.

In other aspects, the treatment can comprise administering to a subject in need thereof a pharmaceutically effective amount of a Toca 511 or an analogue or derivative thereof. In another aspect, the Toca 511 or analogue or derivative is DB107 or an analogue or derivative thereof.

In some aspects, the companion diagnostic test for the treatment is conducted using a SNP (or SNPs) selected from the group consisting rs61530665, a SNP (or SNPs) in linkage disequilibrium with rs61530665, and a complementary SNP (or SNPs) thereof.

In other aspects, the companion diagnostic test for the treatment is conducted using a SNP (or SNPs) selected from the group consisting of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a SNP listed in any of Tables 1A-1E, 2 and 3, a complementary SNP (or SNPs) thereof, and a SNP (or SNPs) in linkage disequilibrium therewith.

In other aspects, the companion diagnostic test for the treatment is conducted using rs61530665, or a complementary SNP (or SNPs) thereof.

In some aspects, the companion diagnostic test is conducted for assessing any suitable type of treatment. For example, the companion diagnostic test is conducted for assessing a neoplasm, cancer, or tumor treatment and/or a gene delivery or therapy treatment using a viral vector.
In some aspects, the companion diagnostic test is conducted for assessing a neoplasm, cancer, or tumor treatment. The cancer or tumor can be a brain cancer or tumor, a multiple myeloma, a pancreatic cancer, a liver cancer, a stomach cancer, a breast cancer, a kidney cancer, a lung cancer, a colorectal cancer, a colon cancer, a prostate cancer, an ovarian cancer, a cervical cancer, a skin cancer, an esophagus cancer, a head and neck cancer, a lymphoma, or a leukemia. The cancer or tumor can also be high grade glioma (HGG), e.g., glioma/glioblastoma (GBM) and anaplastic astrocytoma (AA), non-small cell lung cancer (NSCLC), or lymphoma.

In some aspects, the companion diagnostic test is conducted for assessing a gene delivery or therapy treatment using a viral vector. Any suitable viral vector can be used. For example, the viral vector can be a retroviral vector. In some aspects, the retroviral vector is a non-replication competent retroviral vector. In other aspects, the retroviral vector is a replication competent retroviral vector. In other aspects, the retroviral vector is a recombinant replication competent retroviral vector. In other aspects, the replication competent retroviral vector comprises an oncoretroviral vector.

In some aspects, the companion diagnostic test is conducted for assessing a neoplasm, cancer, or tumor treatment, and the neoplasm, cancer, or tumor treatment comprises administering, to a subject in need, an effective amount of an anti-neoplasm, anti-cancer, or anti-tumor prodrug, and delivering, to the subject, an effective amount of an agent that activates the anti-neoplasm, anti-cancer, or anti-tumor prodrug in the subject using a viral vector.

Any suitable anti-neoplasm, anti-cancer, or anti-tumor prodrug can be used in the treatment. For example, the anti-neoplasm, anti-cancer, or anti-tumor prodrug can comprise a pyrimidine analog. In some aspects, the pyrimidine analog is 5-fluorocytosine (5-FC), or a 5-FC analogue or derivative. In other aspects, the pyrimidine analog, 5-FC or a 5-FC analogue or derivative is administered in an extended-release formulation. Other suitable pyrimidine analog or 5-fluorocytosine (5-FC) formulation can also be used. See e.g., WO 2010/002937 A1, US 2011/0268720 A1, U.S. Pat. Nos. 9,320,738 B2, 9,889,133 B2, 10,449,194 B2, and US 2020/0281929 A1.

In some aspects, the companion diagnostic test is conducted for assessing a gene delivery or therapy treatment using a viral vector. In some aspects, the treatment comprises delivering, to the subject, an effective amount of a polynucleotide encoding a polypeptide having cytosine deaminase activity or a cytosine deaminase using a viral vector. Any suitable viral vector can be used. See e.g., WO 2010/002937 A1, US 2011/0268720 A1, U.S. Pat. Nos. 9,320,738 B2, 9,889,133 B2, 10,449,194 B2, US 2020/0281929 A1, WO 2010/045002 A2, US 2011/0217267 A1, U.S. Pat. Nos. 8,722,867 B2, 8,829,173 B2, 9,732,326 B2, 10,035,983 B2, 10,407,666 B2, US 2013/0130986 A1, US 2019/0185821 A1, US 2020/0239858 A1, WO 2010/036986 A2, WO 2011/126864 A2.

For example, the viral vector can be a replicating, non-lytic retroviral vector. In another aspect, the treatment comprises delivering, to the subject, an effective amount of a polypeptide comprising cytosine deaminase activity.

The above treatment, e.g., a combination therapy using pyrimidine analog, 5-FC or a 5-FC analogue or derivative and a polypeptide comprising cytosine deaminase activity, or a polynucleotide encoding a polypeptide having cytosine deaminase activity or a cytosine deaminase using a viral vector, can be used for treating any suitable cancer or tumor. For example, the cancer or tumor can be high grade glioma (HGG), e.g., glioma/glioblastoma (GBM) and anaplastic astrocytoma (AA).

### D. Companion Diagnostic and Other Methods

Another aspect of the invention involves a method, comprising:
a) assaying a biological sample from a subject that is undergoing a treatment or is considered for a treatment for one or more SNPs selected from the group consisting of rs61530665, a SNP (or SNPs) in linkage disequilibrium with rs61530665, and a complementary SNP (or SNPs) thereof; and
b) generating an output based on the assay results of the SNP or SNPs.

In some aspects, the output is a score. In another aspect, the output (e.g., score) is generated with a computer algorithm.

Another aspect of the invention involves using the output of above method to:
a) determine the likely responsiveness of the subject to the treatment; and/or
b) classify (and/or select) the subject as eligible or ineligible for the treatment or continued treatment; and/or
c) determine whether the subject or the population (when samples from a population of subjects are assayed) is likely to benefit from the treatment or continued treatment, and/or whether the subject or the population is likely to experience an adverse effect from the treatment or continued treatment; and/or
d) determine whether the subject should continue to receive the treatment.

In another aspect, the above methods optionally further comprise
obtaining a biological sample from a subject that is undergoing a treatment or is considered for a treatment.

In another aspect, the above methods optionally further comprise
obtaining a biological sample from a subject that is undergoing a treatment or is considered for a treatment.
isolating genomic DNA from the biological sample.

In other aspects, based on the test or assay result or determination, the methods further comprise one or more of:
a) subjecting the subject to the treatment,
b) continuing the treatment on the subject,
c) not recommending the treatment on the subject,
d) modifying the treatment on the subject, or
e) withdrawing the subject from the treatment.

In some aspects, in the above methods the assay involves one or more SNPs that are selected from the group consisting of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a SNP listed in any of Tables 1A-1E, 2 and 3, a complementary SNP (or SNPs) thereof, and a SNP (or SNPs) in linkage disequilibrium therewith.

In other aspects, in the above methods the assay involves one or more SNPs that are selected from the group consisting of rs61530665, a SNP in linkage disequilibrium with rs61530665 with the D′ value of linkage equilibrium of the SNP being 1 or with the r² value between the SNP and rs61530665 being 1.

In other aspects, in the above methods the assay involves a SNP listed in Table 3, or a complementary SNP (or SNPs) thereof.

In other aspects, in the above methods the assay involves a SNP that is rs61530665, or a complementary SNP (or SNPs) thereof.

In some aspects, in the above methods the assay involves a SNP or SNPs that comprise a sequence (or sequences) set forth in any of the sequences set forth in SEQ ID NOs: 1-25, a complementary sequence (or sequences) thereof, or a sequence (or sequences) in linkage disequilibrium therewith.

In other aspects, in the above methods the assay involves a SNP or SNPs that comprise any of the sequences set forth in SEQ ID NOs: 1-11, 15-21, and 25, a complementary sequence (or sequences) thereof, or a sequence (or sequences) in linkage disequilibrium therewith.

In other aspects, in the above methods the assay involves a SNP or SNPs that comprise any of the sequences set forth in SEQ ID NOs: 1-5, a complementary sequence (or sequences) thereof, or a sequence (or sequences) in linkage disequilibrium therewith.

In other aspects, in the above methods the assay involves a SNP or SNPs that comprise a sequence set forth in SEQ ID NO: 1, a complementary sequence thereof, or a sequence in linkage disequilibrium therewith.

In other aspects, in the above methods, the SNP or the SNPs can be assayed using any suitable assay technique or format, including the techniques described herein.

In some aspects, the methods are conducted for assessing any suitable type of treatment. For example, the methods can be conducted for assessing a neoplasm, cancer, or tumor treatment and/or a gene delivery or therapy treatment using a viral vector.

In some aspects, the methods are conducted for assessing a neoplasm, cancer, or tumor treatment. The cancer or tumor can be a brain cancer or tumor, a multiple myeloma, a pancreatic cancer, a liver cancer, a stomach cancer, a breast cancer, a kidney cancer, a lung cancer, a colorectal cancer, a colon cancer, a prostate cancer, an ovarian cancer, a cervical cancer, a skin cancer, an esophagus cancer, a head and neck cancer, a lymphoma, or a leukemia. The cancer or tumor can also be high grade glioma (HGG), e.g., glioma/glioblastoma (GBM) and anaplastic astrocytoma (AA), non-small cell lung cancer (NSCLC) or lymphoma.

In some aspects, the methods are conducted for assessing a gene delivery or therapy treatment using a viral vector. Any suitable viral vector can be used. For example, the viral vector can be a retroviral vector. In some aspects, the retroviral vector is a non-replication competent retroviral vector. In other aspects, the retroviral vector is a replication competent retroviral vector. In other aspects, the retroviral vector is a recombinant replication competent retroviral vector. In other aspects, the replication competent retroviral vector comprises an oncoretroviral vector.

In some aspects, the methods are conducted for assessing a neoplasm, cancer, or tumor treatment, and the neoplasm, cancer, or tumor treatment comprises administering, to a subject in need, an effective amount of an anti-neoplasm, anti-cancer, or anti-tumor prodrug, and delivering, to the subject, an effective amount of an agent that activates the anti-neoplasm, anti-cancer, or anti-tumor prodrug in the subject using a viral vector.

In some aspects, any suitable anti-neoplasm, anti-cancer, or anti-tumor prodrug is used in the treatment. For example, the anti-neoplasm, anti-cancer, or anti-tumor prodrug can comprise a pyrimidine analog. In some aspects, the pyrimidine analog is 5-fluorocytosine (5-FC), or a 5-FC analogue or derivative. In other aspects, the pyrimidine analog, 5-FC or a 5-FC analogue or derivative is administered in an extended-release formulation. Other suitable pyrimidine analogs or 5-fluorocytosine (5-FC) formulations can also be used. See e.g., WO 2010/002937 A1, US 2011/0268720 A1, U.S. Pat. Nos. 9,320,738 B2, 9,889,133 B2, 10,449,194 B2, and US 2020/0281929 A1.

In some aspect, the methods are conducted for assessing a gene delivery or therapy treatment using a viral vector. In some aspects, the treatment comprises delivering, to the subject, an effective amount of a polynucleotide encoding a polypeptide having cytosine deaminase activity or a cytosine deaminase using a viral vector. Any suitable viral vector can be used. See e.g., WO 2010/002937 A1, US 2011/0268720 A1, U.S. Pat. Nos. 9,320,738 B2, 9,889,133 B2, 10,449,194 B2, US 2020/0281929 A1, WO 2010/045002 A2, US 2011/0217267 A1, U.S. Pat. Nos. 8,722,867 B2, 8,829,173 B2, 9,732,326 B2, 10,035,983 B2, 10,407,666 B2, US 2013/0130986 A1, US 2019/0185821 A1, US 2020/0239858 A1, WO 2010/036986 A2, WO 2011/126864 A2. For example, the viral vector can be a replicating, non-lytic retroviral vector. In other aspects, the treatment comprises delivering, to the subject, an effective amount of a polypeptide comprising cytosine deaminase activity.

In some aspects, the methods are conducted for assessing a combination therapy or treatment using pyrimidine analog, 5-FC or a 5-FC analogue or derivative and a polypeptide comprising cytosine deaminase activity, or a polynucleotide encoding a polypeptide having cytosine deaminase activity or a cytosine deaminase using a viral vector. For example, the cancer or tumor can be high grade glioma (HGG), e.g., glioma/glioblastoma (GBM) and anaplastic astrocytoma (AA). In some aspects, the combination therapy or treatment comprises surgically removing the brain cancer or tumor, and delivering to the subject, an effective amount of a polynucleotide encoding a polypeptide having cytosine deaminase activity or a cytosine deaminase using a viral vector into the tissues lining the hole where the cancer or tumor was (a resection margin or surgical margin).

In some aspects, the treatment assessed by the above methods further comprises administering another medication to the subject for treating a disease or condition, e.g., administering a standard care for the disease or condition. In some aspects, the treatment assessed by the above methods further comprises administering another medication to a subject for treating a cancer or tumor. In some aspects, the treatment assessed by the above methods suppresses cancer or tumor growth, migration, and/or metastasis.

Another aspect of the invention involves a method of identifying a new biomarker using one or more SNPs selected from the group consisting of rs61530665, a SNP (or SNPs) in linkage disequilibrium with rs61530665, and a complementary SNP (or SNPs) thereof.

In some aspects, the above methods are conducted using one or more SNPs selected from the group consisting of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a SNP listed in any of Tables 1A-1E, 2 and 3, a complementary SNP (or SNPs) thereof, and a SNP (or SNPs) in linkage disequilibrium therewith.

In other aspects, the above are conducted using one or more SNPs selected from the group consisting of rs61530665, a SNP in linkage disequilibrium with rs61530665 with the D′ value of linkage equilibrium of the SNP being 1 or with the r² value between the SNP and rs61530665 being 1.

In some aspects, the above methods are conducted using a SNP that is rs61530665, or a complementary SNP (or SNPs) thereof.

The above methods can be conducted to identify any suitable type of a new biomarker, e.g., a DNA, a RNA, a polypeptide, a siRNA or another form of biomarker.

Another aspect of the invention involves a method of identifying a drug target using one or more SNPs selected from the group consisting of rs61530665, a SNP (or SNPs) in linkage disequilibrium with rs61530665, and a complementary SNP (or SNPs) thereof.

In some aspects, the above methods are conducted using one or more SNPs selected from the group consisting of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a SNP listed in any of Tables 1A-1E, 2 and 3, a complementary SNP (or SNPs) thereof, and a SNP (or SNPs) in linkage disequilibrium therewith.

In other aspects, the above are conducted using one or more SNPs selected from the group consisting of rs61530665, a SNP in linkage disequilibrium with rs61530665 with the D′ value of linkage equilibrium of the SNP being 1 or with the r² value between the SNP and rs61530665 being 1.

In other aspects, in the above methods the assay involves a SNP that is rs61530665, or a complementary SNP (or SNPs) thereof.

In some aspects, the drug target is identified based on a biological pathway related to the one or more SNPs.

### E. Methods of Treatment

Another aspect of the invention involves a method for treating a neoplasm, cancer, or tumor in a subject, comprising administering an effective amount of an anti-neoplasm, anti-cancer, or anti-tumor prodrug, and delivering an effective amount of an agent that activates the anti-neoplasm, anti-cancer, or anti-tumor prodrug using a viral vector to a subject in need thereof and that has at least one minor allele (or possesses genotypes with at least one minor allele) for one or more SNPs selected from the group consisting of rs61530665, a SNP (or SNPs) in linkage disequilibrium with rs61530665, and a complementary SNP (or SNPs) thereof.

In some aspects, the subject has at least one minor allele (or possesses genotypes with at least one minor allele) for one or more SNPs that are selected from the group consisting of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a SNP listed in any of Tables 1A-1E, 2 and 3, a complementary SNP (or SNPs) thereof, and a SNP (or SNPs) in linkage disequilibrium therewith.

In other aspects, the subject has at least one minor allele (or possesses genotypes with at least one minor allele) for one or more SNPs that are selected from the group consisting of rs61530665, a SNP in linkage disequilibrium with rs61530665 with the D′ value of linkage equilibrium of the SNP being 1 or with the r² value between the SNP and rs61530665 being 1, e.g., a SNP listed in Table 3, or a complementary SNP (or SNPs) thereof.

In other aspects, the subject has at least one minor allele (or possesses genotypes with at least one minor allele) for a SNP that is rs61530665, or a complementary SNP (or SNPs) thereof.

In some aspects, the subject has at least one minor allele (or possesses genotypes with at least one minor allele) for the SNP or SNPs that comprise a sequence set forth in any of the sequences set forth in SEQ ID NOs: 1-25, a complementary sequence (or sequences) thereof, or a sequence (or sequences) in linkage disequilibrium therewith.

In other aspects, the subject has at least one minor allele (or possesses genotypes with at least one minor allele) for the SNP or SNPs that comprise any of the sequences set forth in SEQ ID NOs: 1-11, 15-21, and 25, a complementary sequence (or sequences) thereof, or a sequence (or sequences) in linkage disequilibrium therewith.

In other aspects, the subject has at least one minor allele (or possesses genotypes with at least one minor allele) for the SNP or SNPs that comprise any of the sequences set forth in SEQ ID NOs: 1-5, a complementary sequence (or sequences) thereof, or a sequence (or sequences) in linkage disequilibrium therewith.

In other aspects, the subject has at least one minor allele (or possesses genotypes with at least one minor allele) for the SNP or SNPs that comprise a sequence set forth in SEQ ID NO: 1, a complementary sequence thereof, or a sequence in linkage disequilibrium therewith.

In some aspects, the present methods are used for treating any suitable neoplasm, cancer, or tumor in a subject. For example, the cancer or tumor can be a brain cancer or tumor, a multiple myeloma, a pancreatic cancer, a liver cancer, a stomach cancer, a breast cancer, a kidney cancer, a lung cancer, a colorectal cancer, a colon cancer, a prostate cancer, an ovarian cancer, a cervical cancer, a skin cancer, an esophagus cancer, a head and neck cancer, a lymphoma, or a leukemia. In some aspects, the cancer or tumor can be high grade glioma (HGG), e.g., glioma/glioblastoma (GBM) and anaplastic astrocytoma (AA), non-small cell lung cancer (NSCLC) or lymphoma.

Any suitable viral vector can be used in the methods. For example, the viral vector can be a retroviral vector. See e.g., WO 2010/002937 A1, US 2011/0268720 A1, U.S. Pat. Nos. 9,320,738 B2, 9,889,133 B2, 10,449,194 B2, US 2020/0281929 A1, WO 2010/045002 A2, US 2011/0217267 A1, U.S. Pat. Nos. 8,722,867 B2, 8,829,173 B2, 9,732,326 B2, 10,035,983 B2, 10,407,666 B2, US 2013/0130986 A1, US 2019/0185821 A1, US 2020/0239858 A1, WO 2010/036986 A2, WO 2011/126864 A2.

In some aspects, the retroviral vector is a non-replication competent retroviral vector. In other aspects, the retroviral vector is a replication competent retroviral vector. In other aspects, the retroviral vector is a recombinant replication competent retroviral vector. In other aspects, the replication competent retroviral vector comprises an oncoretroviral vector. In other aspects, the retroviral vector can be a replicating, non-lytic retroviral vector.

Any suitable anti-neoplasm, anti-cancer, or anti-tumor prodrug can be used in the methods. For example, the anti-neoplasm, anti-cancer, or anti-tumor prodrug can comprise a pyrimidine analog. In some aspects, the pyrimidine analog is 5-fluorocytosine (5-FC), or a 5-FC analogue or derivative. In other aspects, the pyrimidine analog, 5-FC or a 5-FC analogue or derivative is administered in an extended-release formulation. Other suitable pyrimidine analog or 5-fluorocytosine (5-FC) formulation can also be used. See e.g., WO 2010/002937 A1, US 2011/0268720 A1, U.S. Pat. Nos. 9,320,738 B2, 9,889,133 B2, 10,449,194 B2, and US 2020/0281929 A1.

In some aspects, the methods comprise delivering, to the subject, an effective amount of a polynucleotide encoding a polypeptide having cytosine deaminase activity or a cytosine deaminase using a viral vector. Any suitable viral vector can be used in the methods. For example, the viral vector can be a replicating, non-lytic retroviral vector. In another example, the retroviral vector can be a recombinant replication competent retroviral vector.

In some aspects, the methods comprise delivering, to the subject, an effective amount of a polypeptide having cytosine deaminase activity.

In some aspects, the methods are conducted as a combination therapy or treatment using pyrimidine analog, 5-FC or a 5-FC analogue or derivative and a polypeptide comprising cytosine deaminase activity, or a polynucleotide encoding a polypeptide having cytosine deaminase activity or a cytosine deaminase using a viral vector, and can be used for treating any suitable cancer or tumor. For example, the cancer or tumor can be high grade glioma (HGG), e.g., glioma/glioblastoma (GBM) and anaplastic astrocytoma (AA). In some aspects, the combination therapy or treatment comprises surgically removing the brain cancer or tumor, and delivering to the subject, an effective amount of a polynucleotide encoding a polypeptide having cytosine deaminase activity or a cytosine deaminase using a viral vector into the tissues lining the hole where the cancer or tumor was (a resection margin or surgical margin).

In some aspects, the methods further comprise administering another medication to a subject for treating a cancer or tumor.

In some aspects, the methods are conducted to suppress cancer or tumor growth, migration and/or metastasis.

In some aspects, the methods are conducted for treating a neoplasm, cancer, or tumor in any suitable subject. For example, the methods can be conducted for treating a neoplasm, cancer, or tumor in a mammal, e.g., a human or a non-human mammal.

Another aspect of the invention involves the use of an effective amount of: a) an anti-neoplasm, anti-cancer, or anti-tumor prodrug, e.g., a pyrimidine analog, 5-fluorocytosine (5-FC), or a 5-FC analogue or derivative; and b) a polypeptide having cytosine deaminase activity, or a viral vector configured for delivering a polynucleotide encoding a polypeptide having cytosine deaminase activity or a cytosine deaminase, for the manufacture of a medicament for treating a neoplasm, cancer, or a tumor in a subject in need thereof and has at least one minor allele (or possesses genotypes with at least one minor allele) for one or more SNPs selected from the group consisting of rs61530665, a SNP (or SNPs) in linkage disequilibrium with rs61530665, and a complementary SNP (or SNPs) thereof.

### F. Methods for Delivering a Payload to a Subject

Another aspect of the invention involves a method for delivering a payload to a subject, comprising delivering an effective amount of a payload, using a viral vector that comprises a polynucleotide encoding the payload, to a subject in need and that has at least one minor allele (or possesses genotypes with at least one minor allele) for one or more SNPs selected from the group consisting of rs61530665, a SNP (or SNPs) in linkage disequilibrium with rs61530665, and a complementary SNP (or SNPs) thereof.

In some aspects, the methods are conducted for delivering any suitable payload to a subject. For example, the payload can comprise a polypeptide. In some aspects, the polypeptide can be an antibody, a hormone, a regulatory molecule, an enzyme, or a functional fragment or derivative thereof. In other aspects, the payload can comprise a polynucleotide. In other aspects, the polynucleotide can be a DNA or an RNA, e.g., a small interfering RNA (siRNA).

In some aspects, the methods are conducted for any suitable purpose or application. For example, the methods can be conducted for treating or preventing a disease or a disorder, and the payload is delivered to treat or prevent a disease or a disorder in a subject. The payload can comprise a drug, a prodrug or a molecule for converting a prodrug to a drug. In another example, the methods can be conducted for diagnosis, prognosis, risk assessment or monitoring of a disease or a disorder, and the payload is delivered for diagnosis, prognosis, risk assessment or monitoring of a disease or a disorder in a subject. The payload can comprise a signaling, detection or imaging moiety, a precursor thereof, or a molecule for converting a precursor of a signaling, detection or imaging moiety into the signaling, detection or imaging moiety.

In some aspect, the methods are conducted for treating or preventing any suitable disease or a disorder. For example, the disease or a disorder can be selected from the group consisting of an infectious disease, a parasitic disease, a neoplasm, a disease of the blood and blood-forming organs, a disorder involving the immune mechanism, endocrine, nutritional and metabolic diseases, a mental and behavioral disorder, a disease of the nervous system, a disease of the eye and adnexam, a disease of the ear and mastoid process, a disease of the circulatory system, a disease of the respiratory system, a disease of the digestive system, a disease of the skin and subcutaneous tissue, a disease of the musculoskeletal system and connective tissue, a disease of the genitourinary system, pregnancy, childbirth and the puerperium, a condition originating in the perinatal period, a congenital malformation, a deformation, a chromosomal abnormality, an injury, a poisoning, a consequence of external causes, and an external cause of morbidity and mortality.

In some aspects, the disease or disorder is a neoplasm, cancer, or tumor. In some aspects, the cancer or tumor is a brain cancer or tumor, a multiple myeloma, a pancreatic cancer, a liver cancer, a stomach cancer, a breast cancer, a kidney cancer, a lung cancer, a colorectal cancer, a colon cancer, a prostate cancer, an ovarian cancer, a cervical cancer, a skin cancer, an esophagus cancer, a head and neck cancer, a lymphoma, or a leukemia. For example, the cancer or tumor can be high grade glioma (HGG), e.g., glioma/glioblastoma (GBM) and anaplastic astrocytoma (AA), non-small cell lung cancer (NSCLC) or lymphoma.

In some aspects, the subject has at least one minor allele (or possesses genotypes with at least one minor allele) for one or more SNPs that are selected from the group consisting of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a SNP listed in any of Tables 1A-1E, 2 and 3, a complementary SNP (or SNPs) thereof, and a SNP (or SNPs) in linkage disequilibrium therewith.

In other aspects, the subject has at least one minor allele (or possesses genotypes with at least one minor allele) for one or more SNPs that are selected from the group consisting of rs61530665, a SNP in linkage disequilibrium with rs61530665 with the D′ value of linkage equilibrium of the SNP being 1 or with the r² value between the SNP and rs61530665 being 1, e.g., a SNP listed in Table 3, or a complementary SNP (or SNPs) thereof.

In other aspects, the subject has at least one minor allele (or possesses genotypes with at least one minor allele) for a SNP that is rs61530665, or a complementary SNP (or SNPs) thereof.

In some aspects, the subject has at least one minor allele (or possesses genotypes with at least one minor allele) for the SNP or SNPs that comprise a sequence set forth in any of the sequences set forth in SEQ ID NOs: 1-25, a complementary sequence (or sequences) thereof, or a sequence (or sequences) in linkage disequilibrium therewith.

In other aspects, the subject has at least one minor allele (or possesses genotypes with at least one minor allele) for the SNP or SNPs that comprise any of the sequences set forth in SEQ ID NOs: 1-11, 15-21, and 25, a complementary sequence (or sequences) thereof, or a sequence (or sequences) in linkage disequilibrium therewith.

In other aspects, the subject has at least one minor allele (or possesses genotypes with at least one minor allele) for the SNP or SNPs that comprise any of the sequences set forth in SEQ ID NOs: 1-5, a complementary sequence (or sequences) thereof, or a sequence (or sequences) in linkage disequilibrium therewith.

In other aspects, the subject has at least one minor allele (or possesses genotypes with at least one minor allele) for the SNP or SNPs that comprise a sequence set forth in SEQ ID NO: 1, a complementary sequence thereof, or a sequence in linkage disequilibrium therewith.

Any suitable viral vector can be used in the methods. For example, the viral vector can be a retroviral vector. See e.g., WO 2010/002937 A1, US 2011/0268720 A1, U.S. Pat. Nos. 9,320,738 B2, 9,889,133 B2, 10,449,194 B2, US 2020/0281929 A1, WO 2010/045002 A2, US 2011/0217267 A1, U.S. Pat. Nos. 8,722,867 B2, 8,829,173 B2, 9,732,326 B2, 10,035,983 B2, 10,407,666 B2, US 2013/0130986 A1, US 2019/0185821 A1, US 2020/0239858 A1, WO 2010/036986 A2, WO 2011/126864 A2.

In some aspects, the retroviral vector is a non-replication competent retroviral vector. In other aspects, the retroviral vector is a replication competent retroviral vector. In other aspects, the retroviral vector is a recombinant replication competent retroviral vector. In other aspects, the replication competent retroviral vector comprises an oncoretroviral vector. In other aspects, the viral vector can be a replicating, non-lytic retroviral vector.

In some aspect, the methods further comprise administering, to the subject, an additional agent for treating, preventing, diagnosis, prognosis, risk assessment or monitoring of a disease or a disorder.

In some aspects, the delivering efficiency of the payload to a subject that has at least one minor allele (or possesses genotypes with at least one minor allele) for one or more SNPs selected from the group consisting of rs61530665, a SNP (or SNPs) in linkage disequilibrium with rs61530665, and a complementary SNP (or SNPs) thereof, is enhanced or improved as compared to the delivering efficiency of the payload to a comparable subject that does not have the corresponding at least one minor allele.

In some aspect, the method is conducted for delivering a payload to any suitable subject. For example, the subject can be a mammal, e.g., a human or a non-human mammal.

Another aspect of the invention involves the an use of an effective amount of a viral vector that comprises a polynucleotide encoding a payload for the manufacture of a medicament for treating or preventing a disease or a disorder in a subject in need thereof or prevention and has at least one minor allele (or possesses genotypes with at least one minor allele) for one or more SNPs selected from the group consisting of rs61530665, a SNP in linkage disequilibrium with rs61530665 with the D′ value of linkage equilibrium of the SNP being equal to or greater than about 0.900 and/or with the r² value between the SNP and rs61530665 being equal to or greater than about 0.800, and a complementary SNP (or SNPs) thereof.

### G. Further Exemplary Aspects

Another aspect of the invention involves one or more genomic biomarkers that correlate with different responses (e.g., efficacy, adverse effect, and other end points) among patients receiving a cancer treatment regime, such as DB107, for treating diseases such as glioma/glioblastoma and other cancers. The biomarker or biomarkers can be used in companion diagnostic tests which can help to predict drug responses and apply drugs only to those who will be benefited, and/or exclude those who might have negative outcome and/or adverse effects due to the treatment.

Another aspect of the invention involves a panel of biomarkers comprising a SNPs selected from the group consisting of rs61530665 and other SNPs such as those from Table 1A, Table 1B, Table 2, or Table 3, or complementary sequences thereof, and/or sequences in linkage disequilibrium therewith. In some aspects, the biomarkers may comprise the nucleotide sequences set forth in SEQ ID NOs: 1-25 for example, SEQ ID NO: 1 or complementary sequences thereof, and/or sequences in linkage disequilibrium therewith.

Another aspect of the invention involves a reagent for the assessment of the biomarkers described herein, which may comprise one or more molecules for assaying the SNP. In some aspects, the molecules may be oligonucleotides or polypeptides. In some aspects, the oligonucleotides may comprise the nucleotide sequences set forth in SEQ ID NOs: 1-25, for example, SEQ ID NO: 1, or complementary sequence (or sequences) thereof.

In some aspects, the SNP may be assayed by a technique described herein (e.g., PCR).

Another aspect of the invention involves is a kit for the assessment of a panel of isolated biomarkers, which comprises the reagent described herein.

In some aspects, the biomarker or biomarkers comprise one or more SNPs selected from the group consisting of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a complementary SNP (or SNPs) thereof, and a SNP (or SNPs) in linkage disequilibrium therewith.

In some aspects, the kit further comprises instructions for using the biomarker to conduct a companion diagnostic test.

Another aspect of the invention involves a companion diagnostic test for a treatment using a panel of isolated biomarkers comprising one or more SNPs selected from the group consisting of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a complementary SNP (or SNPs) thereof, and a SNP (or SNPs) in linkage disequilibrium therewith.

In some aspects, the companion diagnostic test comprises:
a) obtaining a biological sample from a subject that is undergoing a treatment or is considered for a treatment;
b) isolating genomic DNA from the biological sample;
c) assaying the panel of biomarkers;
d) generating an output with a computer algorithm based on the assay results of the panel of biomarkers; and/or
e) determining the likely responsiveness of the subject to the treatment.

Another aspect of the invention involves a panel of isolated biomarkers associated and/or linked with two, three, four or more of the SNPs described herein, for example, those in Table 1A, Table 1B, Table 1C, Table 1D, Table 1E, Table 2 and Table 3.

Another aspect of the invention involves a companion diagnostic test for a treatment using one or more isolated biomarkers associated and/or linked with one, two, three, four or more of the SNPs described herein, for example, those in Table 1A, Table 1B, Table 1C, Table 1D, Table 1E, Table 2 and Table 3.

Another aspect of the invention involves a method of prognosticating responsiveness of a subject to a disease treatment using the companion diagnostic test described herein. In some aspects, the treatment may comprise a therapeutic regimen using DB107. In some aspects, the disease is selected from the group consisting of glioblastoma, lung cancer, prostate cancer, and breast cancer. In some aspects, the method is used for selecting a patient who is likely to benefit from the treatment and/or excluding a patient who is likely to experience an adverse effect from the treatment.

Another aspect of the invention involves a method of identifying a new biomarker using the panel of isolated biomarkers described herein. In some aspects, the new biomarker may be a DNA, a RNA, a polypeptide, a siRNA or another form of biomarker.

Another aspect of the invention involves a method of identifying a drug target using the panel of isolated biomarkers described herein. In some aspects, the drug target may be identified based on a biological pathway related to a biomarker, wherein the biological pathway may be selected from the genes related to or regulated by the genomic regions affected by the SNP(s) described herein, such as rs61530665.

With the increasing number of SNPs, such as those identified by the SNP Consortium and the novel methods of genotyping, association studies between DNA variants and disease will increase. Because of the limitations of other linkage methodologies, linkage disequilibrium mapping has become the strategy of choice to map complex diseases through the whole genome.

In another aspect, LD refers to a population association among alleles at two or more loci. It is a measure of co-segregation of alleles in a population. Linkage disequilibrium or allelic association is the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

A marker in linkage disequilibrium can be particularly useful in detecting susceptibility to disease (or another phenotype). The marker may or may not cause the disease. For example, a marker (X) that is not itself a causative element of a disease, but which is in linkage disequilibrium with a gene (including regulatory sequences) (Y) that is a causative element of a phenotype, can be detected to indicate susceptibility to the disease in circumstances in which the gene Y may not have been identified or may not be readily detectable. In another aspect, the term allele frequency corresponds to the fraction of the number of individuals with a given allele over the total number of alleles in the population tested.

In some aspects, linkage disequilibrium (LD) refers to the co-inheritance of alleles (e.g., alternative nucleotides) at two or more different SNP sites at frequencies greater than would be expected from the separate frequencies of occurrence of each allele in a given population. The expected frequency of co-occurrence of two alleles that are inherited independently is the frequency of the first allele multiplied by the frequency of the second allele. Alleles that co-occur at expected frequencies are the to be in “linkage equilibrium.” In contrast, LD refers to any non-random genetic association between allele(s) at two or more different SNP sites, which is generally due to the physical proximity of the two loci along a chromosome. See e.g., U.S. 2008/0299125.

In some aspects, LD can occur when two or more SNPs sites are in close physical proximity to each other on a given chromosome and therefore alleles at these SNP sites will tend to remain unseparated for multiple generations with the consequence that a particular nucleotide (allele) at one SNP site will show a non-random association with a particular nucleotide (allele) at a different SNP site located nearby. Hence, genotyping one of the SNP sites will give almost the same information as genotyping the other SNP site that is in LD. See e.g., US 2008/0299125.

In some aspects, for diagnostic and/or companion diagnostic purposes, if a particular SNP site is found to be useful for diagnosis and/or companion diagnosis, then the skilled artisan would recognize that other SNP sites which are in LD with this SNP site would also be useful for diagnosis and/or companion diagnosis of the condition. Various degrees of LD can be encountered between two or more SNPs with the result being that some SNPs are more closely associated (i.e., in stronger LD) than others. Furthermore, the physical distance over which LD extends along a chromosome differs between different regions of the genome, and therefore the degree of physical separation between two or more SNP sites necessary for LD to occur can differ between different regions of the genome. See e.g., US 2008/0299125.

Methods of analysis of LD and/or identifying loci that are in LD with a known locus are known in the art, for example, as disclosed in US 2004/0072217.

### H. Applications of the Biomarkers

Information generated from genomic biomarkers described herein can be used to determine appropriate dosage and/or treatment regimens for an individual with cancers such as GBM and AA. This knowledge, when applied to dosing or drug selection, can minimize or avoid adverse reactions or therapeutic failure and thus enhance therapeutic efficiency when administering a therapeutic composition, such as DB107.

The biomarkers described herein and their associated SNPs or genes could also be used to predict patient's responses to treatment of other diseases or conditions besides GBM and AA. These diseases include, but are not limited to, lymphoma, lung cancer, prostate cancer, breast cancer, and cancer prevention.

Pharmacogenomics involves tailoring a treatment for a subject according to the subject's genotype as a particular treatment regimen may exert a differential effect depending upon the subject's genotype. For example, based upon the outcome of a prognostic test, a clinician or physician may target pertinent information and preventative or therapeutic treatments to a subject who would be benefited by the information or treatment and avoid directing such information and treatments to a subject who would not be benefited (e.g., the treatment has no therapeutic effect and/or the subject experiences adverse side effects). Information generated from pharmacogenomic biomarkers using a method described herein can be used to determine appropriate dosage and treatment regimens for an individual. This knowledge, when applied to dosing or drug selection, can minimize or avoid adverse reactions or therapeutic failure and thus enhance therapeutic efficiency when administering a therapeutic composition. In some aspects, the pharmacogenomic biomarker may be used to develop a companion diagnostic test.

Therefore, Another aspect of the invention involves a companion diagnostic test using the biomarkers described herein. For example, in another aspect, a physician or clinician may consider applying knowledge obtained in biomarkers using a method described herein, when determining whether to administer a pharmaceutical composition to a subject. In another aspect, a physician or clinician may consider applying such knowledge when determining the dosage, e.g., amount per treatment or frequency of treatments, of a treatment, administered to a patient.

The invention provides methods for assessing or aiding assessment of responsiveness of a subject to treatment. The invention also provides methods for predicting responsiveness or monitoring treatment/responsiveness to a treatment in a subject. The invention provides methods for selecting a subject for treatment and treating the subject. In some aspects, the methods comprise assessing one or more pharmacogenomic biomarkers in a sample obtained from the subject; and predicting, assessing, or aiding assessment of responsiveness of the subject to a treatment based on the genotype of the one or more pharmacogenomic biomarkers.

The following is an example of a pharmacogenomic aspect. A particular treatment regimen can exert a differential effect depending upon the subject's genotype. Where a candidate therapeutic exhibits a significant interaction with a major allele and a comparatively weak interaction with a minor allele (e.g., an order of magnitude or greater difference in the interaction), such a therapeutic typically would not be administered to a subject genotyped as being homozygous for the minor allele, and sometimes not administered to a subject genotyped as being heterozygous for the minor allele. In another example, where a candidate therapeutic is not significantly toxic when administered to subjects who are homozygous for a major allele but is comparatively toxic when administered to subjects heterozygous or homozygous for a minor allele, the candidate therapeutic is not typically administered to subjects who are genotyped as being heterozygous or homozygous with respect to the minor allele.

The methods described herein are applicable to pharmacogenomic methods for preventing, alleviating, or treating conditions such as metabolic disorders, cardiovascular diseases, cancers, etc. For example, a nucleic acid sample from an individual may be subjected to a prognostic test described herein. Where one or more polymorphic variations associated with increased risk of type II diabetes are identified in a subject, information for preventing or treating type II diabetes and/or one or more type II diabetes treatment regimens then may be prescribed to that subject.

In certain aspects, a treatment regimen is specifically prescribed and/or administered to individuals who will most benefit from it based upon their likelihood of responding to a treatment regimen assessed by the methods described herein. Thus, other aspects involve methods for identifying a subject with a high likelihood of responding to a treatment regimen and then prescribing such treatment regimen to individuals identified as having a high likelihood of responding. Thus, certain aspects are directed to a method for treating a subject, comprising: detecting the presence or absence of a pharmacogenomic biomarker associated with responsiveness to a treatment regimen in a nucleotide sequence set forth herein in a nucleic acid sample from a subject, and prescribing or administering the treatment regimen to the subject from whom the sample originated where the presence of a pharmacogenomic biomarker associated with responsiveness to the treatment regimen is detected in the nucleotide sequence.

The treatment sometimes is preventative (e.g., is prescribed or administered to reduce the probability that a disease condition arises or progresses), sometimes is therapeutic, and sometimes delays, alleviates, or halts the progression of a disease condition. Any known preventative or therapeutic treatment for alleviating or preventing the occurrence of a disorder may be prescribed and/or administered.

Pharmacogenomics methods also may be used to analyze and predict a response to a drug or a prodrug. For example, if pharmacogenomics analysis indicates a likelihood that an individual will respond positively to a treatment with a particular drug or a prodrug, the drug or a prodrug may be administered to the individual. Conversely, if the analysis indicates that an individual is likely to respond negatively (or not respond at all) to treatment with a particular drug or a prodrug, an alternative course of treatment may be prescribed. The response to a therapeutic treatment can be predicted in a background study in which subjects in any of the following populations are genotyped: a population that responds favorably to a treatment regimen, a population that does not respond significantly to a treatment regimen, and a population that responds adversely to a treatment regimen (e.g., exhibits one or more side effects). These populations are provided as examples and other populations and subpopulations may be analyzed. Based upon the results of these analyses, a subject is genotyped to predict whether he or she will respond favorably to a treatment regimen, not respond significantly to a treatment regimen, or respond adversely to a treatment regimen.

The comparisons and/or calculations for predicting, assessing, or aiding assessment can be carried out in any convenient manner appropriate to the type of measured value and/or reference value for the pharmacogenomic biomarkers at issue. The process of comparing or calculating may be manual or it may be automatic (such as by a machine including computer-based machine). As will be apparent to those of skill in the art, replicate genotyping may be taken for the pharmacogenomic biomarkers.

Other aspects involve a method of prognosticating responsiveness of a subject to a treatment using the companion diagnostic test described herein. The tests described herein also are applicable to clinical drug trials. In some aspects, the pharmacogenomic biomarkers can be used to stratify or select a subject population for a clinical trial. The pharmacogenomic biomarkers can, in some aspects, be used to stratify individuals that may exhibit a toxic response to a treatment from those that will not. In other aspects, the pharmacogenomic biomarkers can be used to separate those that will be non-responders from those who will be responders. The pharmacogenomic biomarkers described herein can be used in pharmacogenomic-based design and in managing the conduct of a clinical trial.

Thus, Another aspect of the invention involves a method of selecting an individual for inclusion in a clinical trial of a treatment or drug comprising the steps of:
(a) obtaining a nucleic acid sample from an individual;
(b) determining the identity of a polymorphic variation which is associated with a positive response to the treatment or the drug, or at least one polymorphic variation which is associated with a negative response to the treatment or the drug in the nucleic acid sample, and
(c) including the individual in the clinical trial if the nucleic acid sample contains the polymorphic variation associated with a positive response to the treatment or the drug or if the nucleic acid sample lacks the polymorphic variation associated with a negative response to the treatment or the drug.

In addition, the methods described herein for selecting an individual for inclusion in a clinical trial of a treatment or drug encompass methods with any further limitation described in this disclosure, or those following, specified alone or in any combination. The including step (d) optionally comprises administering the drug or the treatment to the individual if the nucleic acid sample contains the polymorphic variation associated with a positive response to the treatment or the drug or the nucleic acid sample lacks the biallelic marker associated with a negative response to the treatment or the drug.

### I. Additional Biomarkers or Drug Targets

Other aspects involve a method for identifying polymorphic variants proximal to the biomarkers described herein. In some aspects, the proximal polymorphic variant identified sometimes is a publicly disclosed polymorphic variant, which for example, sometimes is published in a publicly available database. In other aspects, the polymorphic variant identified is not publicly disclosed and is discovered using a known method, including, but not limited to, sequencing a region surrounding the identified pharmacogenomic biomarker in a group of nucleic samples. Thus, multiple polymorphic variants proximal to a biomarker are identified using this method.

The proximal polymorphic variant often is identified in a region surrounding the biomarker. In certain aspects, this surrounding region is about 50 kb flanking the biomarker (e.g., about 50 kb 5′ of the first polymorphic variant and about 50 kb 3′ of the first polymorphic variant), and the region sometimes is composed of shorter flanking sequences, such as flanking sequences of about 40 kb, about 30 kb, about 25 kb, about 20 kb, about 15 kb, about 10 kb, about 7 kb, about 5 kb, or about 2 kb 5′ and 3′ of the biomarker. In other aspects, the region comprises longer flanking sequences, such as flanking sequences of about 75 kb, about 150 kb, about 300 kb, about 600 kb, about 1,200 kb, about 2,000 kb, about 4,000 kb, about, or about 10,000 kb 5′ and 3′ of the biomarker.

In certain aspects, polymorphic variants are identified iteratively. For example, a first proximal polymorphic variant is identified using the methods described above and then another polymorphic variant proximal to the first proximal polymorphic variant is identified (e.g., publicly disclosed or discovered) and the presence or absence of an association of one or more other polymorphic variants proximal to the first proximal polymorphic variant is determined.

The methods described herein are useful for identifying or discovering additional polymorphic variants that may be used to further characterize a gene, region, or loci associated with a condition, a disease, or a disorder. For example, allelotyping or genotyping data from the additional polymorphic variants may be used to identify a functional mutation or a region of linkage disequilibrium. In certain aspects, polymorphic variants identified or discovered within a region comprising the biomarker are genotyped, and it can be determined whether those polymorphic variants are in linkage disequilibrium with the biomarker. The size of the region in linkage disequilibrium with the biomarker also can be assessed using these genotyping methods. Thus, in another aspect, described herein are methods for determining whether a polymorphic variant is in linkage disequilibrium with a biomarker, and such information can be used in prognosis/diagnosis methods described herein.

Additionally, genes may be identified that are in proximity to the biomarkers, and their functions analyzed. Genes with functions that are directly or indirectly related to the relevant phenotype, or other genes in the same cellular pathway, may be targets for further analysis with the relevant phenotype, and new biomarkers may be identified.

Further provided herein is a method of developing novel therapeutic agents and/or identifying a novel drug or drug target using the biomarkers described herein. In some aspects, the biomarkers and their associated SNPs or genes could gain insight of the underlying biological pathways or mechanisms underlying the studied phenotypes, such as efficacy, adverse effect, or other endpoints, thus they may help us identify new drug targets to treat cancers.

### J. Reagents and Kits

Other aspects involve kits, chips, devices, and/or assays, and the preparation thereof, for use in accordance with aspects described herein. In some aspects, the assay, chip, device, and/or kit comprises primers and/or probes to detect genetic signature of SNPs such the ones listed in Table 1A, Table 1B, Table 2 and Table 3. In some aspects, the assay, chip, device, and/or kit comprises a plurality of primers and/or probes. Such methods can include instruments and instructions that a subject can use to obtain a sample, e.g., of buccal cells or blood, without the aid of a health care provider.

Some aspects involve the development of computer algorithm which will convert the test results generated from the measurement of the genomic biomarkers into a score, which will be used to determine in whether an individual should receive the therapeutic invention, such as DB107 treatment.

Another aspect of the invention involves diagnostic kits based on the biomarkers described above, which can be used to predict individual's response to the corresponding drug. In some aspects, the test kits include devices and instructions that a subject can use to obtain a sample, e.g., of buccal cells or blood, without the aid of a health care provider.

For use in the applications described or suggested above, kits, or articles of manufacture are also provided by the invention. In some aspect, the kits comprise at least one reagent (e.g., a primer(s), probe(s), or combination of a primer(s) and probe(s)) specific for genotyping a biomarker (or biomarkers) described herein and optionally further includes instructions for carrying out a method described herein.

Some aspects involve compositions and kits comprising primers, which allow the specific amplification of a polynucleotide or polynucleotides described herein or of any specific parts thereof, and probes that selectively or specifically hybridize to nucleic acid molecules of the invention or to any part thereof. In some aspects, the probes are labeled with a detectable marker, such as, for example, a radioisotope, fluorescent compound, bioluminescent compound, a chemiluminescent compound, metal chelator, or enzyme. In some aspect, the probes and primers are used to detect the presence of a polynucleotide (or polynucleotides) in a sample and/or as a means for detecting cell expressing proteins encoded by the polynucleotides. As will be understood by the skilled artisan, various primers and probes may be prepared based on the sequences provided herein and used effectively to amplify, clone, and/or determine the presence and/or levels of genomic DNAs.

In some aspect, the reagent comprises:
i) one or more primers that allow for amplification of a specific part of or all of the SNP or SNPs being assayed; and,
ii) one or more probes that selectively or specifically hybridize to a specific part of or all of the SNP or SNPs being assayed.

In some aspect, the reagent comprises:
i) a plurality of primers that allow for amplification of a specific part of or all of the SNP or SNPs being assayed; and,
ii) a plurality of probes that selectively or specifically hybridize to a specific part of or all of the SNP or SNPs being assayed.

Some aspects involve a reagent for detecting one or more SNPs selected from the group consisting of rs61530665, a SNP in linkage disequilibrium with rs61530665 with the D′ value of linkage equilibrium of the SNP being equal to or greater than about 0.900 and/or with the r² value between the SNP and rs61530665 being equal to or greater than about 0.800, and a complementary SNP or SNPs thereof, the reagent, comprising:
i) one or more primers that allow for amplification of a specific part of or all of the SNP or SNPs being detected; and,
ii) one or more probes that selectively or specifically hybridize to a specific part of or all of the SNP or SNPs being detected.

In some aspects, a plurality of primers is 2 probes. Other examples include 3, 4, 5, 6, 7, 8, 9, 10, or more primers.

In some aspects, a plurality of probes is 2 probes. Other examples include 3, 4, 5, 6, 7, 8, 9, 10, or more probes.

In some aspects, a technique similar to the TaqMan SNP Genotyping Assay is used to assay a sample for the presence of one or more SNPs. In these aspects, a pair of primers is typically used—e.g., a forward primer and a reverse primer. These forward and reverse primers are specific to the sequence to be amplified (i.e., the sequence that contains the SNP). In these aspects, a pair of probes is typically used. The first probe is configured to detect the first allele sequence. The second probe is configured to detect the second allele sequence. These probes are typically labeled with two different fluorescent dyes. The substantial presence of one dye indicates homozygosity for that allele. A mixture of signals generally indicates heterozygosity. Also, in these aspects a polymerase such as Taq polymerase is typically use for the amplification.

In some aspect, each SNP assayed comprises 4, 8, 10, 15, 20, 25, 30, 50, 60, 100, 300, or 500 nucleotides on either side of the SNP position.

In some aspects, the SNPs assayed by the reagent(s) described herein are one or more SNPs selected from the group consisting of rs61530665, rs74574131, rs111690409, rs72868158, rs72868159, rs113068018, rs11097446, rs10010009, rs35916271, rs61342109, rs28668115, rs28410673, rs10005604, rs10017318, rs10017322, rs4859454, rs4859698, rs11097449, rs144594843, rs200298082, rs28398292, rs28372129, rs10222847, rs7694149, rs17002139, a SNP listed in any of Tables 1A-1E, 2 and 3, a complementary SNP (or SNPs) thereof, and a SNP (or SNPs) in linkage disequilibrium therewith.

In some aspects, the SNPs assayed by the reagent(s) described herein are one or more SNPs selected from the group consisting of rs61530665, a SNP in linkage disequilibrium with rs61530665 with the D′ value of linkage equilibrium of the SNP being 1 or with the r2 value between the SNP and rs61530665 being 1.

In some aspects, the SNP assayed by the reagent(s) described herein is a SNP that is rs61530665, or a complementary SNP (or SNPs) thereof.

In some aspects, the kit comprises reagents for detecting the presence of polypeptides. Such reagents may be antibodies or other binding molecules that specifically bind to the polypeptide(s). In some aspects, the antibodies or binding molecules are capable of distinguishing a structural variation to the polypeptide as a result of polymorphism, and thus can used for genotyping. In some aspect, the antibodies or binding molecules are labeled with a detectable marker, such as, for example, a radioisotope, fluorescent compound, bioluminescent compound, a chemiluminescent compound, metal chelator or enzyme. Other reagents for performing binding assays, such as ELISA, may be included in the kit.

In some aspects, the kits comprise reagents for genotyping at least two, at least three, at least five, at least ten, or more biomarkers. In some aspects, the kits further comprise a surface or substrate (such as a microarray) for capture probes for detecting of amplified nucleic acids.

In some aspect, the kits further comprise a carrier means being compartmentalized to receive in close confinement one or more container means such as vials, tubes, and the like, each of the container means comprising one of the separate elements to be used in the method. For example, one of the container means comprises a probe that is optionally detectably labeled. In some aspects, such probe is a polynucleotide specific for a biomarker. In some aspect, where the kit utilizes nucleic acid hybridization to detect the target nucleic acid, the kit also has containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

In some aspects, the kit comprises the container described above and one or more other containers comprising materials desirable from a commercial and user standpoint, including buffers, diluents, filters, needles, syringes, and package inserts with instructions for use. In some aspects, a label is present on the container to indicate that the composition is used for a specific therapy or non-therapeutic application, and optionally indicates directions for either in vivo or in vitro use, such as those described above.

In some aspects, the kit further comprises a set of instructions and materials for preparing a tissue or cell sample and preparing nucleic acids (such as genomic DNA) from the sample.

Some aspects involve a variety of compositions suitable for use in performing methods of the invention, which may be used in kits. For example, the invention provides surfaces, such as arrays that can be used in such methods. In some aspects, an array comprises individual or collections of nucleic acid molecules useful for detecting pharmacogenomic biomarkers described herein. For example, in some aspects, the invention comprises a series of discretely placed individual nucleic acid oligonucleotides or sets of nucleic acid oligonucleotide combinations that are hybridizable to a sample comprising target nucleic acids, whereby such hybridization is indicative of genotypes of the pharmacogenomic biomarkers of the invention.

Several techniques are well-known in the art for attaching nucleic acids to a solid substrate such as a glass slide. One method is to incorporate modified bases or analogs that contain a moiety that is capable of attachment to a solid substrate, such as an amine group, a derivative of an amine group or another group with a positive charge, into nucleic acid molecules that are synthesized. The synthesized product is then contacted with a solid substrate, such as a glass slide, which is coated with an aldehyde or another reactive group which will form a covalent link with the reactive group that is on the amplified product and become covalently attached to the glass slide. Other methods, such as those using amino propyl silica surface chemistry, are also known in the art, as disclosed at cmt.corning.com and cmgm.stanford.edu/pbrown1.

Attachment of groups to oligonucleotides that can be later converted to reactive groups is also possible using methods known in the art. Any attachment to nucleotides of oligonucleotides will become part of oligonucleotide, which can then be attached to the solid surface of the microarray. Amplified nucleic acids can be further modified, such as through cleavage into fragments or by attachment of detectable labels, prior to or following attachment to the solid substrate, as required and/or permitted by the techniques used.

### Examples. Biomarkers for Predicting Toca511 and Toca FC Responsiveness

Toca 511 (vocimagene amiretrorepvec), in combination with Toca FC (extended-release flucytosine, (5-FC)), is an investigational combination product currently under development for the treatment of high grade glioma (HGG) and other solid tumors. Toca 511 is a gammaretroviral replicating vector derived from a cloned Moloney murine leukemia virus (MLV) that encodes an optimized yeast cytosine deaminase (CD) gene. This approach can overcome the shortcomings of previous gene therapy protocols, by using a retroviral replicating vector (RRV) to deliver the therapeutic gene, cytosine deaminase (CD), to the tumor cells. It has the following advantages:
Selectivity for tumor cell transduction occurs because the RRV infects only dividing cells and preferentially survives in the immune-suppressed tumor microenvironment.
The replicating virus stably integrates into the genome of cancer cells, which is passed to future generations of the infected cancer cells. These infected cells also produce progeny viruses that can infect other cancer cells.
Initial infection and further spread occur without significant inflammation (Lin 2014). Subsequently, multiple cycles of prodrug therapy selectively cause cancer cell lysis, inflammatory release of tumor antigens, and the potential for anti-tumor immune activation and long-term tumor control.
The CD enzyme converts the prodrug 5-FC to the antineoplastic drug 5-FU in the tumor; this molecule has a very short half-life in blood, and the toxicities seen with systemic administration of 5-FU are not observed.
Toca 511 and 5-FC have a dual mechanism of action, including direct tumor cell killing by 5-FU in both immune competent and immune deficient rodents (Huang 2013, Ostertag 2012, Tai 2005) and anti-cancer immune activation only in immune competent models (Ostertag 2012, Yagiz 2015, Hiraoka 2017, Mitchell 2017). The anti-cancer immune activation, which is observed after 5-FC administration, includes a reduction of immune suppressive myeloid cells, including myeloid-derived immune suppressor cells (MDSC) and tumor associated macrophages (TAM), in the tumor microenvironment followed by an increase in T cell (CD4 and CD8) and B cell subsets, as Toca 511 and 5-FC treated tumors regress.

The original ecotropic envelope gene has been replaced with an amphotropic envelope gene, enabling the virus to infect human cells (Logg 2001). Toca 511 selectively infects dividing tumor cells, integrates into the genome, and replicates due to innate and adaptive immune responses that are defective in malignant cells but intact in normal tissues (Ostertag 2012). The vector and CD gene construct is classified as a prodrug activator form of gene therapy, in which the prodrug activator CD enzyme catalyzes the intracellular conversion of the antifungal drug, flucytosine (5-fluorocytosine, 5-FC), to the antineoplastic drug, 5-fluorouracil (5-FU). 5-FU kills cancer cells and immune-suppressive myeloid cells, such as myeloid-derived suppressor cells and tumor associated macrophages, thereby decreasing one or more of the immune brakes in tumors. Multiple studies have been conducted in both tumor-bearing and non-tumor-bearing mice with Toca 511 doses ranging from 1.7×10³ to 6.3×10⁸ TU/g of brain (hereafter abbreviated as TU/g), with and without subsequent administration of 5-FC. Toca 511 at doses ranging from 1.7×10³ to 4.7×10⁵ TU/g in combination with 5-FC was shown to significantly prolong survival in three distinct mouse brain tumor models in three different mouse strains. Two mouse tumor types and one human glioma cell line were successfully treated in these intracranial (IC) models. A durable, prolonged, and statistically significant survival benefit over the control group was achieved in xenograft immune deficient and immune competent mouse models, consistent with a mechanism of action due to 5-FU induced cancer cell death and, in the immune competent mice, induction of an anti-tumor immune response. In immune competent models, apparent cures can be achieved. Evidence of survival benefit was observed in the human xenograft model at IC Toca 511 doses as low as 1.7×10³ TU/g. In an immune competent syngeneic mouse glioma model, intravenous (IV) administration of Toca 511 over 5 consecutive days (3.3×10⁸ TU/g) followed by cyclic administration of 5-FC resulted in a statistically significant increase in survival compared to the control. Additional preclinical data demonstrate evidence of immune memory resulting in protection from tumor re-challenge, additive efficacy with temozolomide, radiation therapy, and lomustine, suitability of use with bevacizumab, and enhanced infection and expression in some models with sunitinib pretreatment. Intravenous administration of Toca 511 with subsequent 5-FC dosing also showed treatment benefit in a metastatic colon cancer model. An animal model of bladder cancer using a similar vector to Toca 511 in combination with 5-FC was associated with reduction in tumor volume and a survival benefit (Kikuchi 2007).

Tocagen has conducted five multicenter clinical studies of Toca 511 and Toca FC in patients with recurrent HGG, including three Phase 1 ascending dose studies (Tg 511-08-01, Tg 511-11-01, and Tg 511-13-01), each of which evaluated different modes of delivery of Toca 511. Across these initial Phase 1 studies, a total of 127 subjects have been treated at different dose levels of Toca 511 and Toca FC, and different routes of administration of Toca 511. A continuation study (Tg 511-09-01) for the long-term follow-up of subjects treated with Toca 511 in the Phase 1 ascending dose studies is ongoing, to evaluate for delayed adverse events (AEs) in accordance with regulatory requirements for gene transfer studies.

The initial, first-in-human clinical study (Tg 511-08-01, NCT01156584) was an ascending dose study primarily to evaluate the safety and tolerability of increasing doses of Toca 511 administered intratumorally, via stereotactic, transcranial injection and followed by orally administered Toca FC. This study showed that a single intratumoral administration of Toca 511 was well tolerated. Subsequent analysis of excised tumor tissue revealed virus was present in the tumor. In each case, virus was detected at multiple sites in the brain tumor indicating that Toca 511 can survive and spread in these tumors.

The second clinical study (Tg 511-11-01, NCT01470794) evaluated ascending doses of Toca 511 injected into the bed of the resection cavity in subjects with recurrent HGG who were undergoing resection, followed by orally administered Toca FC. Median OS (11.9 months) was substantially prolonged in comparison to historical controls; estimated median OS for subjects at first or second recurrence, no prior bevacizumab exposure, tumor size ≤5 cm, GBM or AA, and in the higher dose cohorts was 14.4 months. Study Tg 511-11-01 served as the basis for the design of the registrational study that is the subject of this clinical study report (CSR).

The third clinical study (Tg 511-13-01, NCT01985256) evaluated the safety and tolerability of increasing doses of Toca 511 administered intravenously with subsequent intracranial delivery at the time of resection followed by orally administered Toca FC in subjects with recurrent HGG who were undergoing planned resection. Overall, IV and IC Toca 511 administration, followed by oral doses of Toca FC, was well tolerated and demonstrated an acceptable safety profile; IV injection followed by IC injection of Toca 511 and orally administered Toca FC was associated with a median OS of 13.6 months.

A maximum tolerated dose (MTD) was not established in any of the ascending dose trials; however, a recommended Phase 2 dose was determined based on preliminary dose response observed in Study Tg 511-11-01 wherein a maximum feasible dose of Toca 511 for injection into the resection cavity wall was determined to be ˜1.3×10⁹ TU (˜4 mL). In the context of these trials, the combined dosing of Toca 511 and Toca FC has been evaluated and found to be well tolerated. Findings from these studies supported initiation of a Phase 2/3 study to evaluate the efficacy and safety of Toca 511 and Toca FC for the treatment of subjects with recurrent HGG (Tg 511-15-01).

Study Tg 511-15-01 (Toca 5), is a multicenter, Phase 2/3 randomized, open label study of Toca 511 and Toca FC (Toca arm) versus standard of care (SOC arm) that comprised investigator's choice of single agent chemotherapy (Lomustine or temozolomide) or bevacizumab administered to subjects undergoing resection for first or second recurrence (including this recurrence) of GBM or AA.

For the Experimental Arm, this study used a volume of Toca 511 of 4 mL (approximately 1.3×10⁹ transducing units (TU)), an amount of virus within the dose found to be well tolerated in phase 2 study Tg 511-11-01. Toca 511 was administered once by making multiple injections (up to 40 injections of 100 μL each) into the walls of the resection cavity immediately following tumor resection. Beginning approximately 6 weeks after tumor resection (provided the subject had sufficiently recovered from surgery), oral Toca FC at a dose of 220 mg/kg/day was taken for 7 days, with the course repeated every 6 weeks. The daily dose of Toca FC was administered in 3 equally divided doses to be taken approximately every 8 hours (Q8h) with food. Treatment could begin later than 6 weeks if the subject was unable to begin treatment due to postoperative condition (e.g., inability to swallow Toca FC). Subjects could continue taking Toca FC until the investigator decided that no further benefit could be derived from treatment, or if intolerance was observed.

Systemic treatment in the control arm (SOC arm) began approximately 6 weeks after tumor resection, providing the subject had sufficiently recovered. Treatment could begin later than 6 weeks if the subject was unable to begin treatment due to their postoperative condition (e.g., inability to receive chemotherapy). Lomustine, temozolomide, or bevacizumab may have begun up to 2 weeks earlier (i.e., 4 weeks post tumor resection). Since there are various treatment alternatives for recurrent GBM or AA, investigators could choose one option from the single agent treatments listed in Table 1. When selecting the treatment, investigators were to take into consideration the subject's prior treatment (e.g., subjects who had received prior lomustine were not to receive it again) and clinical status following surgical resection of the tumor.

This is an open-label study; however, the Sponsor remains blinded to the study results and thus only aggregate data are reported in this Investigator's Brochure. No formal analysis per arm has yet been conducted aside from the periodic analyses reviewed by the Independent Data Monitoring Committee (IDMC). The most frequently reported SAEs (≥5 events, 1.9%), regardless of relationship to study drug, include seizure, brain edema, mental status changes, pulmonary embolism and deep vein thrombosis.

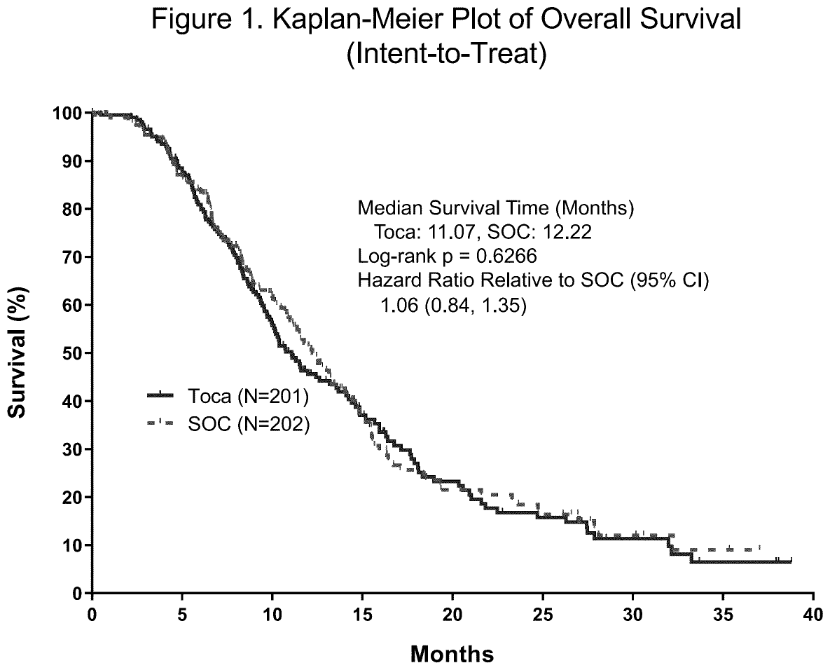

A total of 403 subjects were enrolled into the study. Of these, 259 (64.3%) discontinued from the study due to disease progression, 62 (15.4%) due to withdrawal of consent, and Sponsor decision, and 25 (6.2%) due to Investigator's or Sponsor's decision. A total of 11 subjects (2.7%) discontinued treatment due to AE(s), and 8 (5.6%) completed the study. Other reasons for discontinuation included AE (11 subjects, 2.7%), death (8 subjects, 2.0%), and other (9 subjects, 2.2%). Statistical significance was not observed for the primary endpoint of overall survival (OS) in the ITT population, where the p-value did not meet its nominal significance level of 0.0232 (Table 13). The median OS was 11.07 months (95% CI: 9.86% to 13.54%) for the Toca arm and 12.22 months (95% CI: 10.87% to 14.03%) for the SOC arm (HR=1.06; 95% CI: [0.83, 1.35], Log-rank p=0.6266) (FIG. 1).

In 2020, Denovo Biopharma LLC (Denovo) completed the transfer of ownership of Toca 511 and Toca FC to enable its further clinical development and rename the therapy to DB107. Denovo used its genomewide approach to identifying a novel pharmacogenomic biomarker DGM7 which can be used predict sub-populations of patients more likely to respond to DB107.

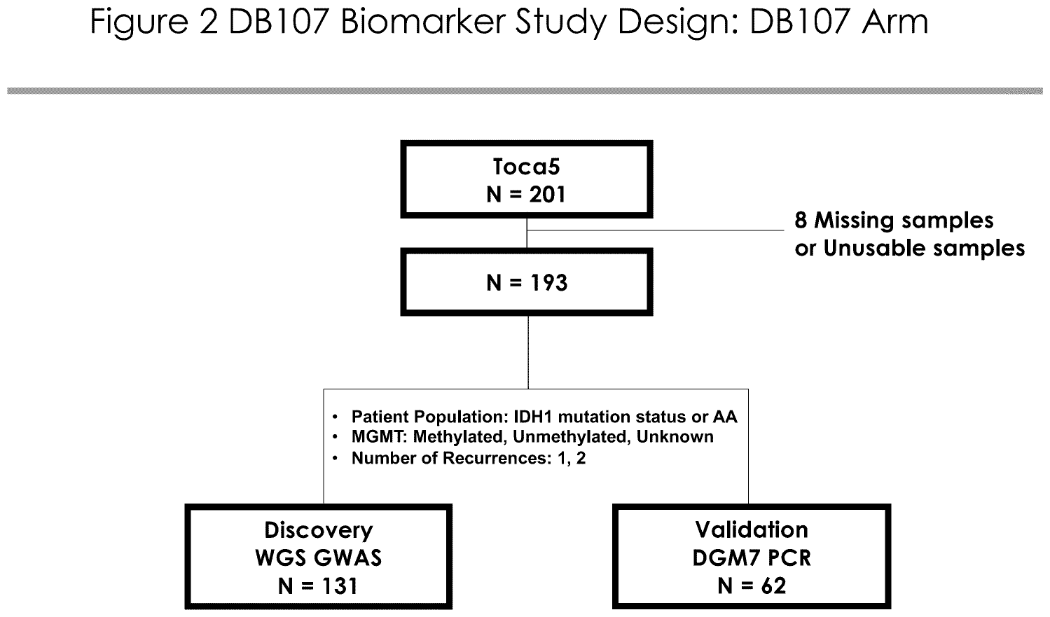

Unlike most oncology biomarker studies focusing on mutations or target protein overexpression in tumor cells, germline genetic polymorphisms also contribute to the various response to the same drug in different patients. Thus, germline DNA samples extracted from blood of patients enrolled in Toca 5 were used to identify pharmacogenetic biomarker for DB107. From the 201 patients in the treatment arm, DNA samples from 193 patients were able to be used in this biomarker study (FIG. 2). In discovery phase, 193 samples from DB107 treatment group were divided into discovery set (N=131) and validation set (N=62) and the two sets were balanced by the following criteria:
1. Patient Population: IDH1 mutation status or AA, WT GBM,
2. MGMT: Methylated, Unmethylated, Unknown,
3. Number of Recurrences: 1, 2.

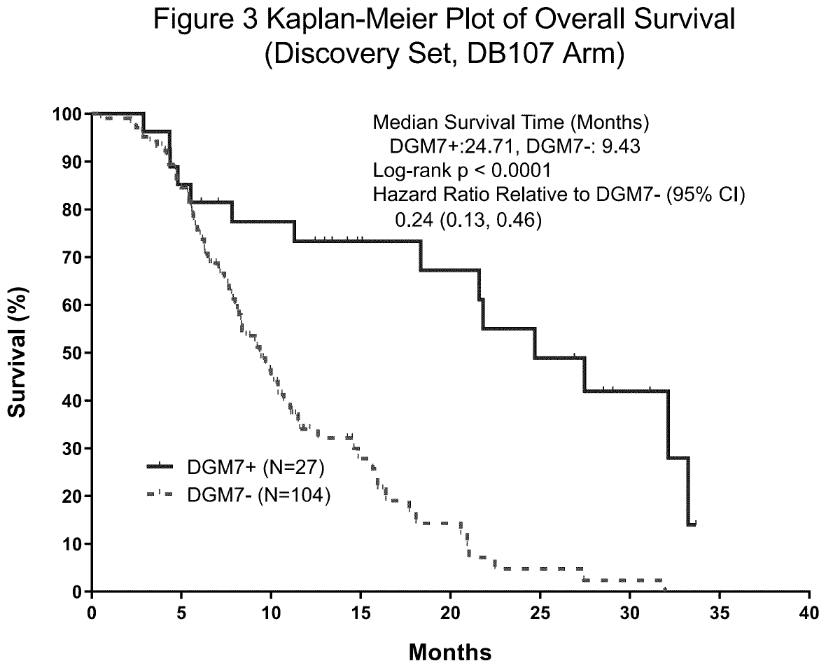

The 131 DNA samples in the discovery set were whole genome sequenced (WGS) at a minimum 10× coverage at Novogene Corporation, Inc, in North America. Sample fastqs were analyzed using Illumina's BaseSpace Dragen Germline v3.7.5 pipeline. After a series of filtering steps to control the qualify, more than 6 million SNPs across the human genome remained for genome-wide association study which was conducted using overall survival as phenotype. SNP rs61530665 was found to have a p-value of 3.879×10⁻⁸, and rs61530665 also exhibited the best Log Rank p-value (<0.0001), best HR (0.24, 95% C.I. 0.13, 0.46), and best median difference (Median Difference 15.28 months, DGM7+: 24.71 months, DGM7−: 9.43 months) in survival for patients possessing genotypes with CT or TT genotypes, when compared to patients carrying CC genotype. DGM7+ was therefore defined as possessing genotypes with at least one minor allele, e.g., genotypes for either CT or TT, and DGM7− was defined as containing the major allele homozygote, e.g., the genotype for CC (FIG. 3).

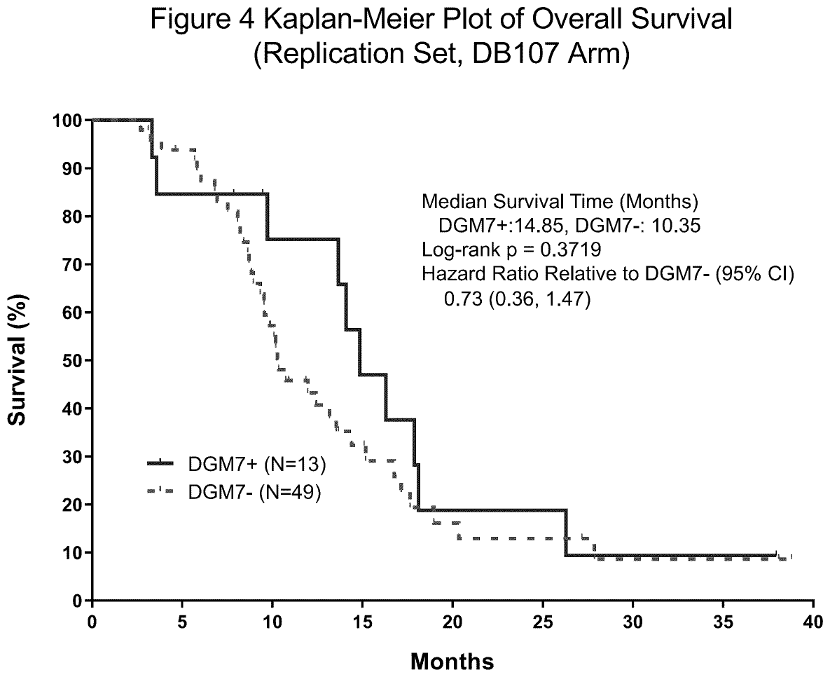

The replication analysis was performed with the remaining 62 samples from the DB107 treatment group using RT-PCR genotyping on DGM7 (Tagman probes and primers purchased from Thermo Fisher). The replication set also showed the clinical benefit of DB107 in survival for DGM7+ patients, when compared to DGM7− with a Log Rank p-value (0.37), HR (0.73, 95% C.I. 0.36, 1.47), and median difference (Median Difference 4.50 months, DGM7+: 14.85 months, DGM7−: 10.35 months) (FIG. 4). Despite the replication set doesn't reach the statistical significance between DGM7+ subjects vs. DGM7− subjects due to the small sample size (Only 13 DGM7+ subjects), it does show the same trend between the two groups.

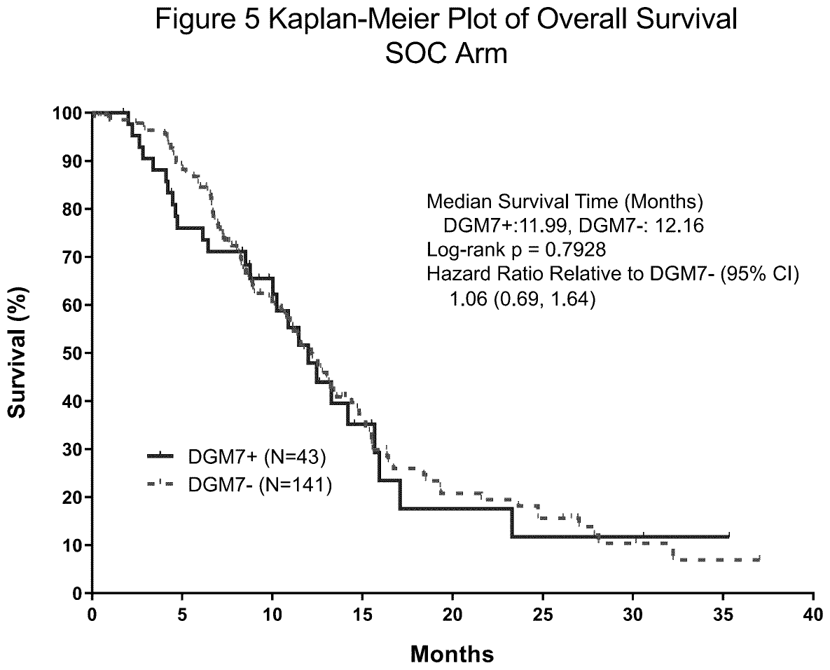

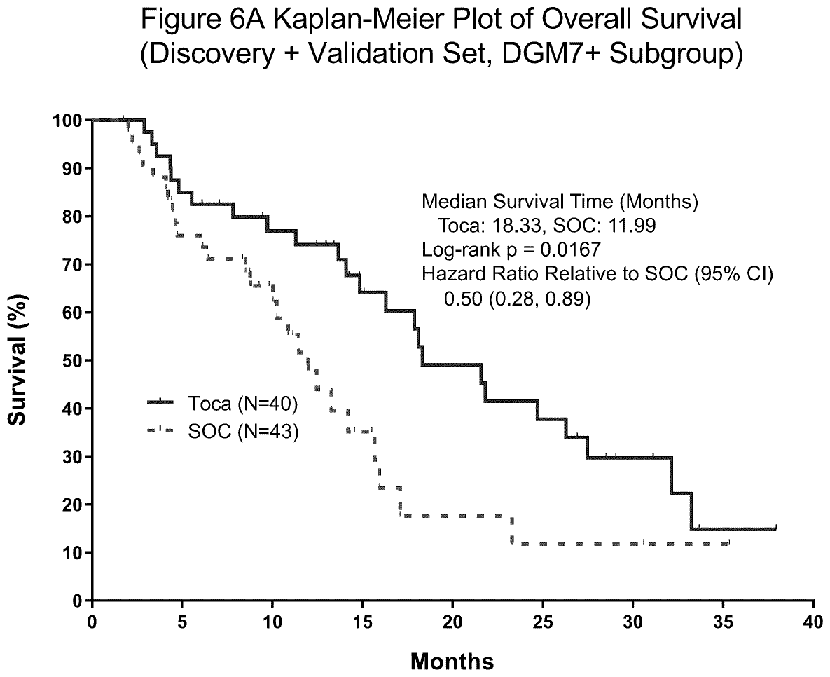

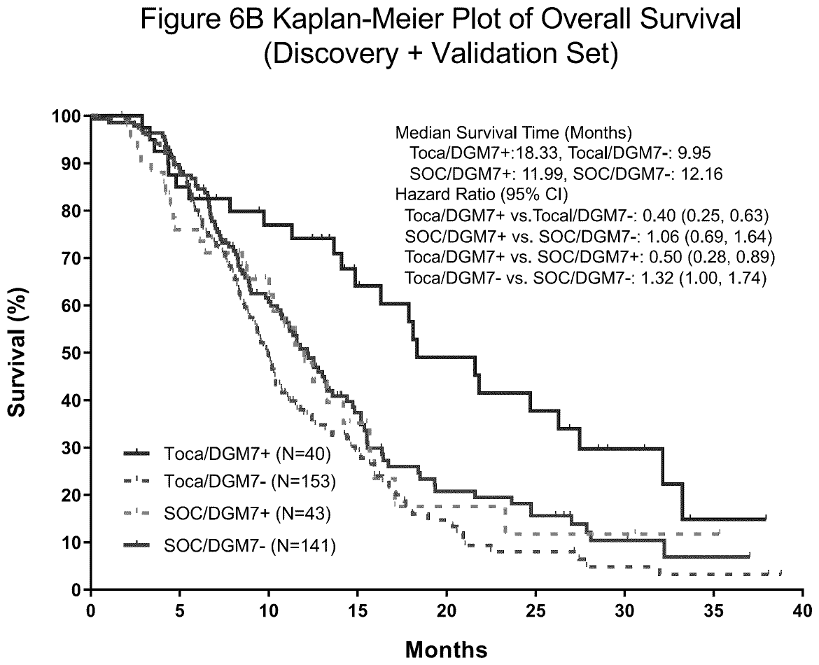

To examine whether rs61530665 is merely a prognostic biomarker for GBM or whether it is specific for DB107, DNA samples from 184 out of 202 subjects from the control arm were available for genotyping at rs61530665 using the Taqman SNP assays. FIG. 5 shows that there is no significant difference in overall survival between patients carrying DGM7+ genotype vs patients carrying DGM7− genotype with a Log Rank p-value (0.79), HR (1.06, 95% C.I. 0.69, 1.64), and median difference (Median Difference −0.17 months, DGM7+: 11.99 months, DGM7−: 12.16 months). Therefore, rs61530665 associated improvement in survival is related to DB107 treatment, and rs61530665 doesn't appears to be a GBM prognosis marker. FIG. 6A shows the direct comparison of the overall survival of DB107 arm vs the SOC arm among DGM7+ subjects with Log Rank p-value 0.0167, and results from DGM7− subjects were also included FIG. 6B. These results demonstrated the superior efficacy of DB107 in DGM7+ subjects comparing to the DGM7− subject as well as to the SOC arm regardless of DGM7 status.

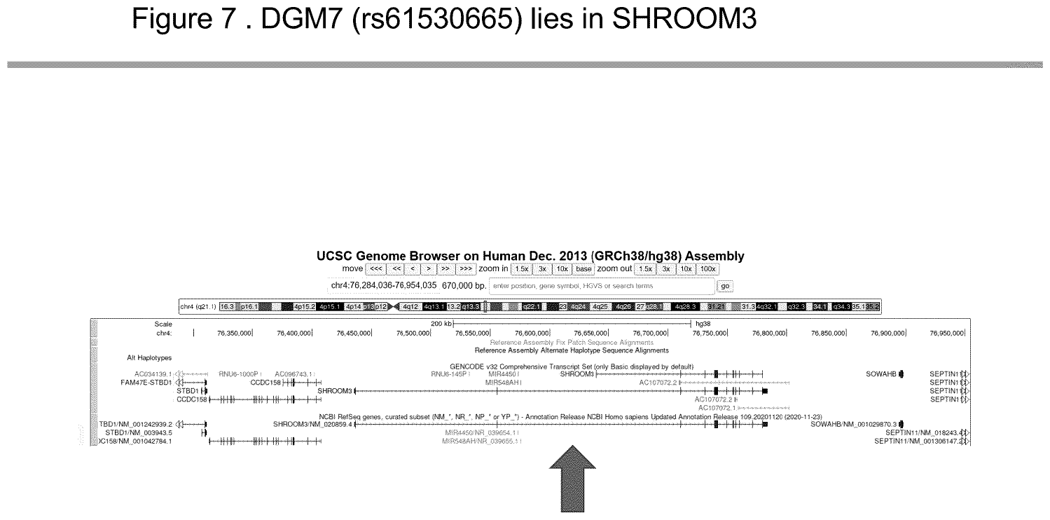

FIG. 7 shows that DGM7 (rs61530665) lies in SHROOM3 gene, which encodes a PDZ-domain-containing protein that belongs to a family of Shroom-related proteins. This protein may be involved in regulating cell shape in certain tissues. A similar protein in mice is required for proper neurulation.

Thus, an aspect involves one or more novel genomic biomarkers that correlate with the activity of DB017. These biomarkers can be used to identify the patients who are most likely to benefit or experience adverse effect from DB107 treatment.

Generally, an isolated SNP-containing nucleic acid molecule comprises one or more SNP positions disclosed by the present invention with flanking nucleotide sequences on either side of the SNP positions. A flanking sequence can include nucleotide residues that are naturally associated with the SNP site and/or heterologous nucleotide sequences. Preferably the flanking sequence is up to about 500, 300, 100, 60, 50, 30, 25, 20, 15, 10, 8, or 4 nucleotides (or any other length in-between) on either side of a SNP position, or as long as the full-length gene or entire protein-coding sequence (or any portion thereof such as an exon).

In another aspect, the biomarkers of the invention are those provided in Table 1A, Table 1B, Table 2 and Table 3, and others complementary to them or in linkage disequilibrium with them:

| SNP | Minor Allele |
|---|---|
| rs61530665 (e.g., as shown in SEQ ID NO: 1): | T |
| SEQ ID NO: 1: TTTTGTATTT TTAGTAGAGA [C > T] | |
| GGGGTTTCAC AGTGTTGGTT | |
| rs74574131 (e.g., as shown in SEQ ID NO: 2): | C |
| SEQ ID NO: 2: GGACTCCCTCTCAAAAAATA [A > C/G/T] | |
| ATACATACATACATACATAC | |
| rs111690409 (e.g., as shown in SEQ ID NO: 3): | Del (-) |
| SEQ ID NO: 3: CTGATCTATTTCTGTTTTAT [G > -] | |
| GAACTAAAGCTTGATTTAGT | |
| rs72868158 (e.g., as shown in SEQ ID NO: 4): | T |
| SEQ ID NO: 4: TACATCAAAATAAGTCACCA [C > A/T] | |
| GTGGGCATCATGAAACCATG | |
| rs72868159 (e.g., as shown in SEQ ID NO: 5): | A |
| SEQ ID NO: 5: GTCATTAGATTATACTCTCC [G > A/T] | |
| GAAAGCTGATTTGATTGATT | |
| rs113068018 (e.g., as shown in SEQ ID NO: 6): | T |
| SEQ ID NO: 6: ATTTGGGTTTTATTTTTAAA [- > T] | |
| GCTGTTGGCATCCGTGTAGA | |
| rs11097446 (e.g., as shown in SEQ ID NO: 7): | A |
| SEQ ID NO: 7: TTTGTACACACATGTTCATA [G > A] | |
| AGCATTATTCACAATAGCCA | |
| rs 10010009 (e.g., as shown in SEQ ID NO: 8): | C |
| SEQ ID NO: 8: CAGATAGTAAATTGCATGTG [T > C] | |
| ATTCTACCACAATTTAAAAA | |
| rs35916271 (e.g., as shown in SEQ ID NO: 9): | TAG |
| SEQ ID NO: 9: GAACTGAGCATGGCAGATCA [- > TAG] | |
| ATTAATAATGCTAGCTGCTG | |
| rs61342109 (e.g., as shown in SEQ ID NO: 10): | G |
| SEQ ID NO: 10: TTAATCTGGAACATGTGCCA [T > G] | |
| CCTTTCTTTAATGAATTGTG | |
| rs28668115 (e.g., as shown in SEQ ID NO: 11): | A |
| SEQ ID NO: 11: GTAGTAGAATGAAGTAGAGG [G > A] | |
| GACGGTCACACAAACCCAGA | |
| rs28410673 (e.g., as shown in SEQ ID NO: 12): | A |
| SEQ ID NO: 12: TAGAATGAAGTAGAGGGGAC [G > A] | |
| GTCACACAAACCCAGAGGGC | |
| rs 10005604 (e.g., as shown in SEQ ID NO: 13): | A |
| SEQ ID NO: 13: CTGAGGTCAGGAGTTCGAGA [C > A] | |
| CAGCCTGGCCAACATGGCAA | |
| rs 10017318 (e.g., as shown in SEQ ID NO: 14): | G |
| SEQ ID NO: 14: ACCTGGTGGAGGGGAGAGGC [A > G/T] | |
| GGAGTTGCAGTGAGCCAAGA | |
| rs 10017322 (e.g., as shown in SEQ ID NO: 15): | G |
| SEQ ID NO: 15: GGCAGGAGTTGCAGTGAGCC [A > G/T] | |
| AGATGATGCCACAGCACTCC | |
| rs4859454 (e.g., as shown in SEQ ID NO: 16): | T |
| SEQ ID NO: 16: CCAAAGACATATTAGAGATA [C > T] | |
| GGGAATTATCTACATAGAGG | |
| rs4859698 (e.g., as shown in SEQ ID NO: 17): | A |
| SEQ ID NO: 17: AAAGACATATTAGAGATACG [G > A/C] | |
| GAATTATCTACATAGAGGGT | |
| rs11097449 (e.g., as shown in SEQ ID NO: 18): | C |
| SEQ ID NO: 18: GGTGGAGGACTAATAAGAAC [A > C] | |
| CATTGTTTCAGAGGCCAAGG | |
| rs 144594843 (e.g., as shown in SEQ ID NO: 19): | GAG |
| SEQ ID NO: 19: GCACTTTTCCCTGGGTCAGC [- > GAG] | |
| AACAGCACCTTATCAGCACC | |
| rs200298082 (e.g., as shown in SEQ ID NO: 20): | Del (-) |
| SEQ ID NO: 20: GCCTTTTATTGCCAAGTATT [A > - | |
| /AA/AAA/AAAA] AAAAAAAAAACAAAAAACTT | |
| rs28398292 (e.g., as shown in SEQ ID NO: 21): | A |
| SEQ ID NO: 21: TTTGGGTGAAGCTTGATTTT [C > A] | |
| TCACCATCATTCTTTCTTTT | |
| rs28372129 (e.g., as shown in SEQ ID NO: 22): | A |
| SEQ ID NO: 22: AATGAGAGATCTGGGCGGGG [G > A] | |
| AAGGGGAGAAAGATATTCCT | |
| rs 10222847 (e.g., as shown in SEQ ID NO: 23): | A |
| SEQ ID NO: 23: TGAAACTTGGTCTCTTGGTC [C > A/T] | |
| AAAAGCTTCCTTTCTTTATA | |
| rs7694149 (e.g., as shown in SEQ ID NO: 24): | A |
| SEQ ID NO: 24: TGACTTGCCCAAAATTACAC [G > A/T] | |
| AAATGGTTACAGCGGGACCA | |
| rs17002139 (e.g., as shown in SEQ ID NO: 25): | G |
| SEQ ID NO: 25: CTCAGGTTCCTGCAGCTACA [A > C/G] | |
| GTGAAGTCCTGAGGTCCCCT | |

The invention includes individual biomarker and biomarker sets. The invention also includes other biomarkers, e.g., SNPs, which have high correlation with the biomarkers, and they could also be used to predict DB107 responses by patients. For examples, those SNPs are in linkage disequilibrium in African populations are provided in Table 1A. The linkage disequilibrium varies in different ethnic groups, for instance the SNPs in linkage disequilibrium in East Asian are shown in Table 1C. Therefore, In another aspect, different SNPs may be used in patients from different ethnic groups to predict DB107 activity and/or responsiveness. Additional predicting SNPs might reside on genes related to the genes that SNPs listed in Table 1A, Table 1B, Table 1C, Table 1D, Table 1E, and Table 2 are associated with. SNPs that are in linkage disequilibrium may be found in various public databases, e.g., 1000 Genomes Project and International HapMap.

The frequency report of rs61530665 in various populations is shown in Table 2. A population is a group (usually a large group) of individuals. Human population samples correspond to samples chosen from a population defined by, for example, ethnicity (population of origin) and geography. For example, population sample could be chosen from different ethnic group such as: African, American, East Asian, European, Asian, and South Asian. Alternatively, human population samples can be selected from an experimental population such as individuals in a diseased population or individuals that react in a particular manner when administered a drug and compared to a control population such as healthy individuals.

TABLE 1A
Variants linked to rs61530665 in 1000GENOMES: Africa

| | | | | | | The |
|---|---|---|---|---|---|---|
| | | | | | | correlation |
| | | | | | D′ value | between a |
| | | | | Distance | of linkage | pair of |
| RS_Number | Location | Alleles | MAF | (bp) | equilibrium | loci: R2 |
| rs10010009 | chr4: 77537988 | (T/C) | 0.3888 | −2201 | 0.9935 | 0.956 |
| rs35916271 | chr4: 77538436 | (−/TAG) | 0.3805 | −1753 | 0.9968 | 0.9904 |
| rs61530665 | chr4: 77540189 | (C/T) | 0.3812 | 0 | 1 | 1 |
| rs61342109 | chr4: 77540337 | (T/G) | 0.3812 | 148 | 1 | 1 |
| rs28668115 | chr4: 77540889 | (G/A) | 0.382 | 700 | 1 | 0.9968 |
| rs28410673 | chr4: 77540893 | (G/A) | 0.379 | 704 | 0.9968 | 0.984 |
| rs10005604 | chr4: 77541017 | (C/A) | 0.379 | 828 | 0.9968 | 0.984 |
| rs10017318 | chr4: 77541156 | (A/G) | 0.382 | 967 | 1 | 0.9968 |
| rs10017322 | chr4: 77541173 | (A/G) | 0.382 | 984 | 1 | 0.9968 |
| rs4859454 | chr4: 77541509 | (C/T) | 0.382 | 1320 | 1 | 0.9968 |
| rs4859698 | chr4: 77541511 | (G/A) | 0.382 | 1322 | 1 | 0.9968 |
| rs11097449 | chr4: 77541666 | (A/C) | 0.382 | 1477 | 1 | 0.9968 |
| rs144594843 | chr4: 77542089 | (−/AGG) | 0.382 | 1900 | 1 | 0.9968 |

TABLE 1B
Variants linked to rs61530665 in 1000GENOMES: American

| | | | | | | The |
|---|---|---|---|---|---|---|
| | | | | | | correlation |
| | | | | | D′ value | between a |
| | | | | Distance | of linkage | pair of |
| RS_Number | Location | Alleles | MAF | (bp) | equilibrium | loci: R2 |
| rs10010009 | chr4: 77537988 | (T/C) | 0.1787 | −2201 | 0.9802 | 0.9514 |
| rs35916271 | chr4: 77538436 | (−/TAG) | 0.1772 | −1753 | 1 | 1 |
| rs61530665 | chr4: 77540189 | (C/T) | 0.1772 | 0 | 1 | 1 |
| rs61342109 | chr4: 77540337 | (T/G) | 0.1772 | 148 | 1 | 1 |
| rs28668115 | chr4: 77540889 | (G/A) | 0.1744 | 700 | 1 | 0.9803 |
| rs28410673 | chr4: 77540893 | (G/A) | 0.1744 | 704 | 1 | 0.9803 |
| rs10005604 | chr4: 77541017 | (C/A) | 0.1744 | 828 | 1 | 0.9803 |
| rs10017318 | chr4: 77541156 | (A/G) | 0.1744 | 967 | 1 | 0.9803 |
| rs10017322 | chr4: 77541173 | (A/G) | 0.1744 | 984 | 1 | 0.9803 |
| rs4859454 | chr4: 77541509 | (C/T) | 0.1758 | 1320 | 0.99 | 0.9705 |
| rs4859698 | chr4: 77541511 | (G/A) | 0.1744 | 1322 | 1 | 0.9803 |
| rs11097449 | chr4: 77541666 | (A/C) | 0.1744 | 1477 | 1 | 0.9803 |
| rs144594843 | chr4: 77542089 | (−/AGG) | 0.1744 | 1900 | 1 | 0.9803 |
| rs28398292 | chr4: 77542689 | (C/A) | 0.1643 | 2500 | 1 | 0.9124 |

TABLE 1C
Variants linked to rs61530665 in 1000GENOMES: East Asia

| | | | | | | The |
|---|---|---|---|---|---|---|
| | | | | | | correlation |
| | | | | | D′ value | between a |
| | | | | Distance | of linkage | pair of |
| RS_Number | Location | Alleles | MAF | (bp) | equilibrium | loci: R2 |
| rs74574131 | chr4: 77527864 | (A/C) | 0.3165 | −12325 | 0.9771 | 0.9547 |
| rs111690409 | chr4: 77528347 | (G/−) | 0.3165 | −11842 | 0.9771 | 0.9547 |
| rs72868158 | chr4: 77528915 | (C/T) | 0.3165 | −11274 | 0.9771 | 0.9547 |
| rs72868159 | chr4: 77529650 | (G/A) | 0.3165 | −10539 | 0.9771 | 0.9547 |
| rs113068018 | chr4: 77531868 | (−/T) | 0.3165 | −8321 | 0.9771 | 0.9547 |
| rs11097446 | chr4: 77537521 | (G/A) | 0.3165 | −2668 | 0.9771 | 0.9547 |
| rs10010009 | chr4: 77537988 | (T/C) | 0.3175 | −2201 | 1 | 0.9954 |
| rs35916271 | chr4: 77538436 | (−/TAG) | 0.3165 | −1753 | 1 | 1 |
| rs61530665 | chr4: 77540189 | (C/T) | 0.3165 | 0 | 1 | 1 |
| rs61342109 | chr4: 77540337 | (T/G) | 0.3165 | 148 | 1 | 1 |
| rs28668115 | chr4: 77540889 | (G/A) | 0.3165 | 700 | 1 | 1 |
| rs28410673 | chr4: 77540893 | (G/A) | 0.3165 | 704 | 1 | 1 |
| rs10005604 | chr4: 77541017 | (C/A) | 0.3165 | 828 | 1 | 1 |
| rs10017318 | chr4: 77541156 | (A/G) | 0.3165 | 967 | 1 | 1 |
| rs10017322 | chr4: 77541173 | (A/G) | 0.3165 | 984 | 1 | 1 |
| rs4859454 | chr4: 77541509 | (C/T) | 0.3165 | 1320 | 1 | 1 |
| rs4859698 | chr4: 77541511 | (G/A) | 0.3165 | 1322 | 1 | 1 |
| rs11097449 | chr4: 77541666 | (A/C) | 0.3165 | 1477 | 1 | 1 |
| rs144594843 | chr4: 77542089 | (−/AGG) | 0.3165 | 1900 | 1 | 1 |
| rs200298082 | chr4: 77542301 | (−/A) | 0.3135 | 2112 | 0.9907 | 0.9681 |
| rs28398292 | chr4: 77542689 | (C/A) | 0.3155 | 2500 | 0.9954 | 0.9863 |
| rs28372129 | chr4: 77545006 | (G/A) | 0.3214 | 4817 | 1 | 0.9774 |
| rs10222847 | chr4: 77547942 | (C/A) | 0.3075 | 7753 | 0.9858 | 0.9323 |
| rs7694149 | chr4: 77551408 | (G/A) | 0.3075 | 11219 | 0.9858 | 0.9323 |

TABLE 1D
Variants linked to rs61530665 in 1000GENOMES: Europe

| | | | | | | The |
|---|---|---|---|---|---|---|
| | | | | | | correlation |
| | | | | | D′ value | between a |
| | | | | Distance | of linkage | pair of |
| RS_Number | Location | Alleles | MAF | (bp) | equilibrium | loci: R2 |
| rs35916271 | chr4: 77538436 | (−/TAG) | 0.0984 | −1753 | 1 | 0.9776 |
| rs61530665 | chr4: 77540189 | (C/T) | 0.0964 | 0 | 1 | 1 |
| rs61342109 | chr4: 77540337 | (T/G) | 0.0984 | 148 | 1 | 0.9776 |
| rs28668115 | chr4: 77540889 | (G/A) | 0.0964 | 700 | 1 | 1 |
| rs28410673 | chr4: 77540893 | (G/A) | 0.0964 | 704 | 1 | 1 |
| rs10005604 | chr4: 77541017 | (C/A) | 0.0994 | 828 | 1 | 0.9668 |
| rs10017318 | chr4: 77541156 | (A/G) | 0.0984 | 967 | 1 | 0.9776 |
| rs10017322 | chr4: 77541173 | (A/G) | 0.0984 | 984 | 1 | 0.9776 |
| rs4859454 | chr4: 77541509 | (C/T) | 0.0974 | 1320 | 1 | 0.9887 |
| rs4859698 | chr4: 77541511 | (G/A) | 0.0964 | 1322 | 1 | 1 |
| rs11097449 | chr4: 77541666 | (A/C) | 0.0964 | 1477 | 1 | 1 |
| rs144594843 | chr4: 77542089 | (−/AGG) | 0.0964 | 1900 | 1 | 1 |
| rs28398292 | chr4: 77542689 | (C/A) | 0.0974 | 2500 | 0.9772 | 0.9441 |
| rs28372129 | chr4: 77545006 | (G/A) | 0.0974 | 4817 | 0.9772 | 0.9441 |
| rs10222847 | chr4: 77547942 | (C/A) | 0.0974 | 7753 | 0.9772 | 0.9441 |
| rs7694149 | chr4: 77551408 | (G/A) | 0.0974 | 11219 | 0.9772 | 0.9441 |
| rs17002139 | chr4: 77576402 | (A/G) | 0.1024 | 36213 | 0.9885 | 0.9142 |

TABLE 1E
Variants linked to rs61530665 in 1000GENOMES: South Asia

| | | | | | | The |
|---|---|---|---|---|---|---|
| | | | | | | correlation |
| | | | | | D′ value | between a |
| | | | | Distance | of linkage | pair of |
| RS_Number | Location | Alleles | MAF | (bp) | equilibrium | loci: R2 |
| rs10010009 | chr4: 77537988 | (T/C) | 0.2014 | −2201 | 0.9935 | 0.9684 |
| rs35916271 | chr4: 77538436 | (−/TAG) | 0.2004 | −1753 | 1 | 0.9873 |
| rs61530665 | chr4: 77540189 | (C/T) | 0.1984 | 0 | 1 | 1 |
| rs61342109 | chr4: 77540337 | (T/G) | 0.1994 | 148 | 1 | 0.9936 |
| rs28668115 | chr4: 77540889 | (G/A) | 0.1984 | 700 | 1 | 1 |
| rs28410673 | chr4: 77540893 | (G/A) | 0.1984 | 704 | 1 | 1 |
| rs10005604 | chr4: 77541017 | (C/A) | 0.1984 | 828 | 1 | 1 |
| rs10017318 | chr4: 77541156 | (A/G) | 0.1984 | 967 | 1 | 1 |
| rs10017322 | chr4: 77541173 | (A/G) | 0.1984 | 984 | 1 | 1 |
| rs4859454 | chr4: 77541509 | (C/T) | 0.1984 | 1320 | 1 | 1 |
| rs4859698 | chr4: 77541511 | (G/A) | 0.1984 | 1322 | 1 | 1 |
| rs11097449 | chr4: 77541666 | (A/C) | 0.1984 | 1477 | 1 | 1 |
| rs144594843 | chr4: 77542089 | (−/AGG) | 0.1984 | 1900 | 1 | 1 |
| rs200298082 | chr4: 77542301 | (−/A) | 0.2025 | 2112 | 0.9935 | 0.9622 |
| rs28398292 | chr4: 77542689 | (C/A) | 0.1984 | 2500 | 1 | 1 |
| rs28372129 | chr4: 77545006 | (G/A) | 0.2045 | 4817 | 1 | 0.9626 |
| rs10222847 | chr4: 77547942 | (C/A) | 0.2025 | 7753 | 0.9935 | 0.9622 |
| rs7694149 | chr4: 77551408 | (G/A) | 0.2025 | 11219 | 0.9935 | 0.9622 |

TABLE 2
rs61530665 Genotype Frequencies in Different Ethnic Groups

| | Allele: | | DGM7- | DGM7 + |
|---|---|---|---|---|
| | Frequency | Genotype: | Fre- | Fre- |
| Population | (count) | Frequency (count) | quency | quency |
| ALL | C: 0.753 (3771) | C\|C: 0.580 (1453) | 0.58 | 0.42 |
| | T: 0.247 (1237) | C\|T: 0.345 (865) | | |
| | | T\|T: 0.074 (186) | | |
| AFR | C: 0.619 (818) | C\|C: 0.386 (255) | 0.386 | 0.614 |
| (African) | T: 0.381 (504) | C\|T: 0.466 (308) | | |
| | | T\|T: 0.148 (98) | | |
| AMR | C: 0.823 (571) | C\|C: 0.689 (239) | 0.689 | 0.311 |
| (American) | T: 0.177 (123) | C\|T: 0.268 (93) | | |
| | | T\|T: 0.043 (15) | | |
| EAS (East | C: 0.684 (689) | C\|C: 0.468 (236) | 0.468 | 0.532 |
| Asia) | T: 0.316 (319) | C\|T: 0.431 (217) | | |
| | | T\|T: 0.101 (51) | | |
| EUR | C: 0.904 (909) | C\|C: 0.815 (410) | 0.815 | 0.185 |
| (Europe) | T: 0.096 (97) | C\|T: 0.177 (89) | | |
| | | T\|T: 0.008 (4) | | |
| SAS | C: 0.802 (784) | C\|C: 0.640 (313) | 0.64 | 0.36 |
| (South | T: 0.198 (194) | C\|T: 0.323 (158) | | |
| Asia) | | T\|T: 0.037 (18) | | |

Table 3 below lists SNPs in linkage disequilibrium with rs61530665 with the D′ value of linkage equilibrium of the SNP being 1 or with the r² value between the SNP and rs61530665 being 1.

TABLE 3
SNPs in linkage disequilibrium with rs61530665
RS_Number
rs10005604
rs10017318
rs10017322
rs11097449
rs144594843
rs28398292
rs28410673
rs28668115
rs35916271
rs4859454
rs4859698
rs61342109
rs61530665
.

Numerous modifications and variations of the invention are possible considering the above teachings. It is therefore to be understood that within the scope of the appended claims, the invention may be practiced otherwise than as specifically described herein.

1. A method of treating a subject that has high grade glioma (HGG) and is being considered for treatment for HGG, comprising: administering:
i) an effective amount of 5-fluorocytosine (5-FC) or a 5-FC analogue or derivative; and,
ii) an effective amount of a polypeptide having cytosine deaminase activity, or a viral vector configured for delivering a polynucleotide encoding a polypeptide having cytosine deaminase activity or a cytosine deaminase;
to a patient known to have one or more SNPs selected from the group consisting of
i) rs61530665,
ii) a SNP in linkage disequilibrium with rs61530665 with the D′ value of linkage equilibrium of the SNP being equal to or greater than about 0.900 and/or with the r2 value between the SNP and rs61530665 being equal to or greater than about 0.800, and
iii) a complementary SNP or SNPs thereof.

2. The method of claim 1, wherein the subject is known to be homozygous or heterozygous for one or more of the minor alleles shown in the following table or a corresponding complement;

| SNP | Minor Allele |
|---|---|
| rs61530665 | T |
| rs74574131 | C |
| rs111690409 | Del (−) |
| rs72868158 | T |
| rs72868159 | A |
| rs113068018 | T |
| rs11097446 | A |
| rs10010009 | C |
| rs35916271 | TAG |
| rs61342109 | G |
| rs28668115 | A |
| rs28410673 | A |
| rs10005604 | A |
| rs10017318 | G |
| rs10017322 | G |
| rs4859454 | TAG |
| rs4859698 | A |
| rs11097449 | C |
| rs144594843 | GAG |
| rs200298082 | Del (−) |
| rs28398292 | A |
| rs28372129 | A |
| rs10222847 | A |
| rs7694149 | A |
| rs17002139 | G |
| | . |

3. The method of claim 1, wherein the subject is known to be homozygous or heterozygous for one or more of the minor alleles shown in the following table or a corresponding complement:

| SNP | Minor Allele |
|---|---|
| rs61530665 | T |
| rs35916271 | TAG |
| rs61342109 | G |
| rs28668115 | A |
| rs28410673 | A |
| rs10005604 | A |
| rs10017318 | G |
| rs10017322 | G |
| rs4859454 | T |
| rs4859698 | A |
| rs11097449 | C |
| rs144594843 | GAG |
| rs28398292 | A |
| | . |

4. The method of claim 1, wherein the subject is known to be homozygous or heterozygous the T allele of rs61530665 or a corresponding complement.

5. The method of claim 1, wherein the HGG is glioblastoma (GBM).

6. The method of claim 1, wherein the HGG is anaplastic astrocytoma (AA).

7. The method of claim 4, wherein the HGG is glioblastoma (GBM).

8. The method of claim 4, wherein the HGG is anaplastic astrocytoma (AA).

9. A companion diagnostic method, comprising:
a) assaying a biological sample from a subject, that is undergoing treatment for high grade glioma (HGG) or is considered for treatment for HGG, for one or more SNPs selected from the group consisting of
i) rs61530665,
ii) a SNP in linkage disequilibrium with rs61530665 with the D′ value of linkage equilibrium of the SNP being equal to or greater than about 0.900 and/or with the r² value between the SNP and rs61530665 being equal to or greater than about 0.800, and
iii) a complementary SNP or SNPs thereof; and
b) generating an output based on the assay results of the SNP or SNPs;
wherein the output is used for at least one of the following:
i) determining the likely responsiveness of the subject to the treatment;
ii) classifying and/or selecting the subject as eligible or ineligible for the treatment or continued treatment;
iii) determining whether the subject is likely to benefit from the treatment or continued treatment, and/or whether the subject is likely to experience an adverse effect from the treatment or continued treatment; and/or
iv) determining whether the subject should continue to receive the treatment.

10. The companion diagnostic of claim 9, wherein:
the subject has HGG and is considered for treatment for HGG; and,
the output is used to classify and/or select the patient for treatment for HGG.

11. The companion diagnostic of claim 9, wherein the HGG is glioblastoma (GBM).

12. The companion diagnostic of claim 9, wherein the HGG is anaplastic astrocytoma (AA).

13. The companion diagnostic of claim 9, wherein:
the subject is a population of subjects and samples from the population of subjects are assayed.

14. The companion diagnostic of claim 9, wherein the one or more SNPs selected from the group consisting of:

| SNP | Minor Allele |
|---|---|
| rs61530665 | T |
| rs74574131 | C |
| rs111690409 | Del (−) |
| rs72868158 | T |
| rs72868159 | A |
| rs113068018 | T |
| rs11097446 | A |
| rs10010009 | C |
| rs35916271 | TAG |
| rs61342109 | G |
| rs28668115 | A |
| rs28410673 | A |
| rs10005604 | A |
| rs10017318 | G |
| rs10017322 | G |
| rs4859454 | TAG |
| rs4859698 | A |
| rs11097449 | C |
| rs144594843 | GAG |
| rs200298082 | Del (−) |
| rs28398292 | A |
| rs28372129 | A |
| rs10222847 | A |
| rs7694149 | A |
| rs17002139 | G |
| | . |

15. The companion diagnostic of claim 9, wherein the one or more SNPs selected from the group consisting of:

| SNP | Minor Allele |
|---|---|
| rs61530665 | T |
| rs35916271 | TAG |
| rs61342109 | G |
| rs28668115 | A |
| rs28410673 | A |
| rs10005604 | A |
| rs10017318 | G |
| rs10017322 | G |
| rs4859454 | T |
| rs4859698 | A |
| rs11097449 | C |
| rs144594843 | GAG |
| rs28398292 | A |
| | . |

16. The companion diagnostic of claim 9, wherein the SNP is the T allele of rs61530665 or a corresponding complement.

17. The companion diagnostic of claim 9, wherein the biological sample is assayed using a reagent, comprising:
i) one or more primers that allow for amplification of a specific part of or all of the SNP or SNPs being assayed; and,
ii) one or more probes that selectively or specifically hybridize to a specific part of or all of the SNP or SNPs being assayed.

18. The companion diagnostic of claim 9, wherein the treatment comprises administering:
a) an effective amount of 5-fluorocytosine (5-FC) or a 5-FC analogue or derivative; and,
b) an effective amount of a polypeptide having cytosine deaminase activity, or a viral vector configured for delivering a polynucleotide encoding a polypeptide having cytosine deaminase activity or a cytosine deaminase.

19. A reagent for detecting one or more SNPs selected from the group consisting of rs61530665, a SNP in linkage disequilibrium with rs61530665 with the D′ value of linkage equilibrium of the SNP being equal to or greater than about 0.900 and/or with the r² value between the SNP and rs61530665 being equal to or greater than about 0.800, and a complementary SNP or SNPs thereof, the reagent, comprising:
i) one or more primers that allow for amplification of a specific part of or all of the SNP or SNPs being detected; and,
ii) one or more probes that selectively or specifically hybridize to a specific part of or all of the SNP or SNPs being detected.

20. The reagent of claim 19, wherein each SNP assayed comprises 4, 8, 10, 15, 20, 25, 30, 50, 60, 100, 300, or 500 nucleotides on either side of the SNP position.

21. A method for delivering a payload to a subject for treating a subject having HGG, comprising delivering an effective amount of a payload, using a viral vector comprising a polynucleotide encoding the payload, to a subject in need, wherein the subject has at least one minor allele for one or more SNPs selected from the group consisting of rs61530665, a SNP in linkage disequilibrium with rs61530665 with the D′ value of linkage equilibrium of the SNP being equal to or greater than about 0.900 and/or with the r² value between the SNP and rs61530665 being equal to or greater than about 0.800, and a complementary SNP or SNPs thereof.
